(19) United States
(12) Patent Application Publication
(10) Pub. No.: US 2025/0099414 A1
(43) Pub. Date: Mar. 27, 2025

(54) COMPOSITIONS AND METHODS FOR TREATING TRIPLE-NEGATIVE BREAST CANCER

(71) Applicant: The Board of Regents of the University of Oklahoma, Norman, OK (US)

(72) Inventors: Roger G. Harrison, Norman, OK (US); Alexis A. WOODWARD, Omaha, NE (US)

(21) Appl. No.: 18/971,835

(22) Filed: Dec. 6, 2024

Related U.S. Application Data
(63) Continuation-in-part of application No. 17/621,385, filed on Dec. 21, 2021.
(60) Provisional application No. 62/867,971, filed on Jun. 28, 2019.

(51) Int. Cl. A61K 31/20; A61K 47/64; A61K 47/68; A61P 35/00

(52) U.S. Cl. CPC A61K 31/20; A61K 47/64; A61K 47/6809; A61P 35/00

(57) ABSTRACT

Therapeutic protein-drug conjugates comprising an annexin protein (e.g., annexin A5) conjugated to a drug payload such as (but not limited to) valproic acid for targeting triple-negative breast cancer (TNBC) cells, and methods of use of the conjugates in the treatment of TNBC in a subject afflicted with TNBC.

### CROSS-REFERENCE TO RELATED APPLICATIONS

The present patent application is a continuation-in-part of U.S. Ser. No. 17/621,385, filed Dec. 21, 2021; which is a national stage filing of PCT Application No. PCT/US2020/039650, filed Jun. 25, 2020; which claims priority to U.S. Provisional Patent Application Ser. No. 62/867,971, filed on Jun. 28, 2019. The entire contents of each of the above-referenced applications are expressly incorporated herein by reference.

### GOVERNMENT SUPPORT

Not Applicable.

### BACKGROUND

The three hormonal receptors that categorize breast cancer subtypes are estrogen receptor (ER), progesterone receptor (PR), and human epidermal growth factor 2 receptor (HER2). When cancers are ER, PR, and/or HER2 positive, patients tend to have a positive response rate to targeted endocrine-based therapies, have lower cell proliferation rates, lower tumor grades, and often have a good prognosis. Triple-negative breast cancer (TNBC) however, is a subset of breast cancers in which the ER, PR, and HER2 hormonal receptors are absent, rendering the triple negative phenotype.

To be categorized as TNBC, less than 1% of the tumor cells must stain positive for ER and PR and must have a staining score of 0 to +1 for HER2 as determined by immunohistochemistry. Although molecular cell staining defines this breast cancer subtype, TNBC is the most heterogenous breast cancer subtype and is further categorized into four different transcriptional subtypes or six different molecular subtypes. TNBC is the most aggressive breast cancer and has a high proliferation index. The aggressive nature of this disease results in high rates of metastasis to the bone, brain, liver, and lungs resulting in poor survival. TNBC makes up 15-20% of all breast cancer cases in the United States of America and has the lowest 5-year survival rate of all breast cancers. According to the National Institutes of Health Surveillance, Epidemiology, and End Results Program (SEER), TNBC 5-year survival is only 77.1% in comparison to hormonal positive breast cancer, which has a 5-year survival rate of 89.9%. Once TNBC regionally or distantly metastasizes, the 5-year survival rates decrease significantly.

Because TNBC is defined by the lack of hormonal receptors ER, PR, and HER2 on the cell surface, targeted endocrine-based therapies are ineffective. Despite many efforts to develop a targeted treatment, TNBC has been categorized as an orphan disease, and the standard of care for TNBC patients remains untargeted-conventional chemotherapy, radiation, and/or surgery. TNBC is treated with a wide range of chemotherapeutic drugs (topoisomerase II inhibitors, DNA alkylating agents, microtubule-targeting taxanes, anti-metabolites, and/or platinum agents), but the drugs are nonselective and induce dose-limiting side effects all over the body (e.g. hair loss, nausea, vomiting, fatigue, hearing loss, weight loss, bone loss, reproductive damage, nephrotoxicity, hepatotoxicity, cardiotoxicity, and/or neurotoxicity). In addition to these dose-limiting side effects, heterogenous tumor barriers such as multi-drug exporters, cancer stem cells, hypoxia, and aberrant signaling pathways all promote singular-and multi-drug resistance. These compounding factors severely limit the therapeutic options for this disease.

It has only been in the last four years that the FDA has approved targeted poly(ADP-ribose) polymerase (PARP) inhibitors, checkpoint inhibitors, and an antibody-drug conjugate for treating TNBC. PARP inhibitors target the genetic instability of cancer and inhibit the repair of single-strand DNA breaks through PARP enzymes. While PARP inhibitors have been advantageous in treating TNBC with germline BRAC mutations, they have been met with challenges in the clinic. PARP inhibitors have high rates of drug resistance, are only effective for the 20% of TNBC patients with BRCA mutations, and increase the overall risk of serious adverse events when compared to traditional chemotherapy.

Checkpoint inhibitors provide a long-lasting anticancer response in patients, and TNBC has been categorized as a tumor with a high density of tumor-infiltrating lymphocytes, especially CD8⁺ T-cells. However, less than 20% of TNBC patients respond to an immune checkpoint blockade therapy, suggesting that there are compounding factors in the tumor microenvironment (TME) that prevent an adaptive antitumor response. TNBC patients have benefited from adding checkpoint inhibitors to their standard-of-care chemotherapy regimens, but when a checkpoint inhibitor is added, 100% of patients experience treatment-related adverse events. The adverse events can be severe and impact the remaining quality of life for patients (e.g., hepatitis, arthralgia, hyper/hypothyroidism, blurred vision, and type 1 diabetes mellitus).

Lastly, only one antibody-drug conjugate has been approved for TNBC. Sacituzumab-govitecan (SG) is a trophoblast cell-surface antigen 2 (Trop-2) targeted humanized antibody linked to the topoisomerase-I inhibitor, SN-38, with a hydrolysable linker. Trop-2 is overexpressed in many cancer types including TNBC and aids in tumorigenesis and anchorage dependent cell growth. SN-38 is the active from of the prodrug, irinotecan, and is linked to the antibody with a property linker that allows for pH-mediated release of the drug in the tumor cell and the TME. While pH-mediated drug release takes advantage of the pH difference between the plasma (pH=7.4) and the TME (pH=4.0-5.0), the spontaneous hydrolysis of the linker results in unloading of SN38 systemically. Additionally, the internalization of the antibody is only 2-fold higher than the control, indicating its cellular internalization maybe inefficient. This suggests the SG antibody-drug conjugate may not be acting as a traditional targeting antibody, but more as an SN-38 prodrug to localize the drug to the tumor microenvironment. In the Phase III ASCENT clinical trial, SG side effects for TNBC patients included neutropenia, leukopenia, diarrhea, anemia, febrile neutropenia, alopecia, and fatigue. When comparing the SG treatment group to traditional chemotherapy, the SG treatment group had side effects that occurred more frequency and presented more severely.

Although PARP inhibitors, checkpoint inhibitors, and SG have provided targeted treatments for TNBC, their therapeutic benefit is constrained by dose-limiting side effects, drug resistance, off target drug unloading, and lack of biomarkers to successfully treat TNBC. Therefore, there is still a need to develop new TNBC targeted therapies to decrease systemic side effects and increase the therapeutic impact of the therapy. It is to such therapeutics that the present disclosure is directed.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 shows effects of ANXA-DM1, free DM1, and free ANXA5 on viability of mouse 4T1 TNBC cells after 72 hours. The concentration of DM1 as the free drug (squares) or in the ANXA5-DM1 conjugate (circles) is shown. IC50 values are 0.85 nM for ANXA5-DM1 vs 320 nM for free DM1 (data presented as mean±SD with n=4).

FIG. 2 shows effects of ANXA5-DM1, free DM1, and free ANXA5 on viability of mouse EMT6 TNBC cells after 72 hours. The concentration of DM1 as the free drug (squares) or in the ANXA5-DM1 conjugate (circles) is shown. IC50 values are 0.21 nM for ANXA5-DM1 vs 28 nM for free DM1 (data presented as mean±SD with n=4).

FIG. 3 shows effects of ANXA5-DM1, free DM1, and free ANXA5 on viability of 100% confluent healthy MCF10 mammary cells after 72 hours. The concentration of DM1 as the free drug (squares) or in the ANXA5-DM1 conjugate (circles) is shown. IC50 values are 160 nM for free DM1, and >5000 nM for ANXA5-DM1 (data presented as mean±SD with n=3).

FIG. 4 shows that there was no statistically significant change in cytotoxicity by ANXA5 treatment as concentration increased for 4T1, EMT6, and MCF10 mammary cells (data presented as mean±SD with n=5 for 4Tl cells and with n=3 for EMT6 and MCF10 cells).

FIG. 5 shows immunogenic cell death (ICD) activity of 10 nM ANXA5-DM1 and DM1 treatment on EMT6 cells as measured through ATP release after 24 hours. There was a significant increase for ANXA5-DM1 treatment compared to free DM1 treatment or the control (data represented as mean±SD n =6). Statistical significance is denoted by **** (p<0.0001).

FIG. 6 shows ICD activity of 10 nM ANXA5-DM1 and DM1 treatment on EMT6 cells as measured through calreticulin surface expression after 24 hours. There was a significant increase in calreticulin surface expression for ANXA5-DM1 treatment compared to free DM1 treatment or the control (data represented as mean±SD n=2 with each sample run in triplicate). Statistical significance is denoted by * (p<0.0332).

FIG. 7 shows ICD activity of 10 nM ANXA5-DM1 and DM1 treatment on EMT6 cells as measured by cytotoxicity after 24 hours. Cytotoxicity activity of EMT6 cells, as measured by cell death, increased significantly for ANXA5-DM1 treatment compared to free DM1 treatment or the control (data represented as mean±SD n=3). Statistical significance is denoted by * (p<0.0332), *** (p<0.0002), and **** (p<0.0001).

FIG. 8 characterizes ANXA5-DM1 by absorbance at 288 nm. Absorbance spectroscopy (OD: 288 nm) of DM1 following spectral correction for ANXA5 for detection of DM1.

FIG. 9 shows binding strength of ANXA5 on EMT6-strain TNBC cells (A) and 4T1-strain TNBC cells (B) with PS externalization. Cells were incubated with 0-20 nM of ANXA5. Total binding (green square with dot-dash line) was measured with the addition of 2 mM Ca²⁺ to promote ANXA5 binding. Nonspecific binding (red triangle with dashed line) was measured with the addition of EDTA to chelate residual Ca2, inhibiting ANXA5 binding. Specific binding (black square with solid line) was obtained by subtracting nonspecific binding from total binding. The dissociation constant of the specific binding was 1.14 nM for EMT6 cells. The dissociation constant of the specific binding was 2.31 nM for 4T1 cells. Data presented as mean±SD (n=3).

FIG. 10 shows an example of an ANAX5-CMB crosslinking synthesis scheme.

FIG. 11 shows the effects of increasing levels of ANXA5-CMB on HUVEC cell viability over 24 hours.

FIG. 12 shows the effects of increasing levels of ANXA5-CMB on MCF10A cell viability over 24 hours.

FIG. 13 shows an example of an ANAX5-VPA crosslinking synthesis scheme. First VPA is activated with EDC. The VPA-EDC reaction generates a reactive O-acylisourea ester intermediate, and once a primary amine (lysine residue) is introduced, the EDC is displaced generating an isourea by-product and the ANXA5-VPA conjugate.

FIG. 14 shows the effects of increasing levels of ANXA5-VPA and free VPA on EMT6 cell viability over 48 hours. Cells were treated with 10⁻⁴ to 10 mM VPA in the ANXA5-VPA conjugate or free VPA in fully supplemented growth medium. The IC50 concentration for ANXA5-VPA was about 0.010 mM, while the IC50 concentration of free VPA could not be calculated. Cell viability was measured via an Alamar Blue assay, and each sample was represented as a percentage of the untreated control (0 mM) and viability was compared to the control (0 mM) after 48 hours. Statistical analysis at each dose was performed with a student's T-test with data represented as mean±SD (n=3).

FIG. 15 shows the effects of increasing levels of ANXA5-VPA and free VPA on 4T1 cell viability over 48 hours. Cells were treated with 10⁻⁴ to 10 mM VPA in the ANXA5-VPA conjugate or free VPA in fully supplemented growth medium. The IC50 concentration for ANXA5-VPA was about 0.010 mM, while the IC50 concentration of free VPA could not be calculated. Cell viability was measured via an Alamar Blue assay, and each sample was represented as a percentage of the untreated control (0 mM) and viability was compared to the control (0 mM) after 48 hours. Statistical analysis at each dose was performed with a student's T-test with data represented as mean±SD (n=3).

### DETAILED DESCRIPTION

Disclosed herein is a therapeutic strategy targeting the aminophospholipid phosphatidylserine (PS) that is expressed on TNBC cells. The therapy is a protein-drug conjugate (bioconjugate) comprising an annexin (e.g., annexin A5, ANXA5) and an anti-cancer drug such as mertansine (a.k.a., DM1, emtansine), chlorambucil (CMB), and/or valproic acid (VPA), which are covalently linked via a cross-linker (e.g., sulfo-SMCC). Without wishing to be bound by theory, the bioconjugate acts against TNBC cells by binding to external PS on the cell surface. After binding to the PS, the bioconjugate is internalized. As the annexin is lysed within the cell, the drug is released and diffuses out of the lysosome causing mitotic catastrophe and cell death. For example, DM1 is a potent anticancer drug that inhibits microtubule formation and induces immunogenic death cell (ICD) both in vitro and in vivo. As shown herein, the ANXA5-DM1 conjugate displays TNBC-specific and enhanced cytotoxicity when compared to free DM1, and importantly, the ANXA5-DM1 conjugate does not have cytotoxic effects against healthy breast cells. It is also shown herein that the ANXA5-DM1 conjugate increased the release or externalization of two hallmark ICD DAMPs. ANXA5 has a high specificity and affinity for externalized PS on TNBC cells as indicated by the low dissociation constant in the low nanomolar range, while no ANXA5-PS binding was observed on healthy MCF10A breast cells.

PS provides an innate biomarker on TNBC cells. In the mammalian plasma membrane, the PS is the only anionic phospholipid and comprises up to 10% of the total lipid content. Despite its abundance, the asymmetry of PS across the plasma membrane is tightly regulated and plays a confounding role in regulating cell death and removal by the innate immune system. In healthy cells, PS exclusively resides within the cytosolic leaflet of the plasma membrane, but as cells become stressed/apoptotic, PS is flipped to the extracellular leaflet where it promotes the removal of the sick cells without activating the adaptive immune system. During neoplastic transformation, the asymmetry of PS across the plasma membrane is also lost, leading to increased exposure of PS on the extracellular leaflet. Cancerous cells have up to seven times more external PS than healthy cells, and PS is highly externalized in metastatic TNBC. However, this increase in PS externalization leads to an immunosuppressive microenvironment, and the cancerous cell survives. Despite its role in cancer progression, PS creates a unique biomarker for targeted chemotherapies.

Proteins of the annexin family are natural binding proteins to PS. Annexins are a diverse family of proteins that play a critical role in the homeostasis of cellular membranes. These small proteins are characterized by their ability to bind to negatively-charged phospholipids in a Ca²⁺-dependent manner. The most notable among annexin family members is annexin A5 (ANXA5), which is a main component in many commercial kits used to detect apoptosis. After binding to PS, ANXA5 enters the cell through a non-receptor-mediated pathway. ANXA5 binds to PS, and the protein crystallizes into a trimer network, bending the plasma membrane inward and forces internalization.

Before further description of embodiments of the present disclosure by way of exemplary drawings, experimentation, results, and laboratory procedures, it is to be understood that the embodiments of the present disclosure are not limited in application to the details of compositions and methods set forth in the following description or illustrated in the drawings, experimentation and/or results. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

The following abbreviations may be used herein.
ALL—Acute lymphocytic leukemia;
AML—Acute myeloid leukemia;
ANOVA—analysis of variance;
ANXA5—AnnexinA5;
ATP—Adenosine triphosphate;
BSA—bovine serum albumin;
° C.—degrees Celsius;
CHL—Chlorambucil;
CLL—Chronic lymphocytic leukemia;
CMB—Chlorambucil;
CML—Chronic myeloid leukemia;
Da—Dalton;
DAMPS—damage-associated molecular patterns;
DI—deionized;
DM1—Drug maytansinoid 1 (Mertansine);
DMSO—Dimethyl sulfoxide;
DNA—Deoxyribonucleic acid;
ELISA—Enzyme-Linked Immunosorbent Assay;
EDC—1-Ethyl-3-[3-dimethylaminopropyl] carbodiimide hydrochloride;
EPR—Enhanced permeability and retention;
FBS—fetal bovine serum;
FDG—2-Fluoro-2-deoxyglucose;
FITC—Fluorescein isothiocyanate;
hEGF—human epidermal growth factor;
HESI—heated electrospray ionization;
ICD—immunogenic cell death;
Ig—Immunoglobulin;
IMAC—immobilized metal affinity chromatography;
IPTG—Isopropyl β-D-1-thiogalactopyranoside;
kDa—kilo Dalton;
LB—Lysogeny broth;
LC—liquid chromatography;
LD50—Median lethal dose;
M—Molarity (unity);
MEGM—mammary epithelial growth medium;
MMP—Matrix metalloproteinase;
mTOR—Mammalian target of rapamycin;
mM—millimolar;
ms—millisecond;
MS—mass spectroscopy;
μM—micromolar;
nM—nanomolar;
OPD—O-phenylenediamine;
PBS—phosphate buffered saline;
PC—Phosphatidylcholine;
PCR—Polymerase Chain Reaction;
PE—Phosphatidylethanolamine;
PMSF—Phenylmethylsulfonyl fluoride;
PS—Phosphatidylserine;
RNA—Ribonucleic acid;
SDS—PAGE-Sodium dodecyl sulfate polyacrylamide gel electrophoresis;
SEC—size exclusion chromatography;
SG—Sacituzumab-govitecan;
Strep-HRP—streptavidin-horseradish peroxidase;
sulfo-NHS—N-hydroxysulfosuccinimide;
sulfo-SMCC—sulfosuccinimidyl 4-(N-maleimidomethyl)cyclohexane-1-carboxylate;
TF—Tissue Factor;
TME—tumor microenvironment;
TNBC—Triple negative breast cancer;
TPCK—N-p-tosyl-L-phenylalanine chloromethyl ketone;
Trop-2—Trophoblast cell-surface antigen 2;
UHPLC—ultra-high-performance liquid chromatography;
UV—Ultraviolet;
VPA—Valproic acid.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo-or polynucleotide chemistry and hybridization described herein are those well-known and commonly used in the art. Standard techniques are used for recombinant DNA, oligonucleotide synthesis, and tissue culture and transformation. Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (4th ed., Cold Spring Harbor Laboratory Press, Cold Spring Harbor, N.Y. (2012) and Coligan et al. Current Protocols in Immunology (Current Protocols, Wiley Interscience (1991-2017)), which are incorporated herein by reference. The nomenclatures utilized in connection with, and the laboratory procedures and techniques of, analytical chemistry, synthetic organic chemistry, molecular and cellular biology, and medicinal and pharmaceutical chemistry described herein are those well-known and commonly used in the art. Standard techniques are used for chemical syntheses, chemical analyses, pharmaceutical preparation, formulation, and delivery, and treatment of patients.

All publications, published patent applications, and issued patents mentioned in the specification are indicative of the level of skill of those skilled in the art to which the presently disclosed inventive concepts pertain. All publications, published patent applications, and issued patents are explicitly incorporated by reference herein to the same extent as if each individual publication, published patent application, or issued patent was specifically and individually indicated to be incorporated by reference.

As utilized in accordance with the present disclosure, the following terms, unless otherwise indicated shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, observer error, and combinations thereof, for example. The term “about” or “approximately,” where used herein when referring to a measurable value such as an amount, a temporal duration, and the like, is meant to encompass, for example, variations of ±20%, or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 80% of the time, at least 90% of the time, at least 91% of the time, at least 92% of the time, at least 93% of the time, at least 94% of the time, at least 95% of the time, at least 96% of the time, at least 97% of the time, at least 98% of the time, or at least 99% of the time.

As noted above, any numerical range listed or described herein is intended to include, implicitly or explicitly, any number or sub-range within the range, particularly all integers, including the end points, and is to be considered as having been so stated. For example, “a range from 1.0 to 10.0” is to be read as indicating each possible number, including integers and fractions, along the continuum between and including 1.0 and 10.0, that is, having a minimum value equal to or greater than 1.0 and a maximum value equal to or less than 10.0, such as, for example, 3.25 to 8.65. Any maximum numerical limitation recited herein is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. Thus, even if a particular data point within the range is not explicitly identified or specifically referred to, it is to be understood that any data points within the range are to be considered to have been specified, and that the inventor(s) possessed knowledge of the entire range and the points within the range.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, and each integer up to and including 50, as well as the fractions 1.1, 1.2, 1.3, 1.4, 1.5, etc., 2.1, 2.2, 2.3, 2.4, 2.5, etc., and so forth. Reference to a series of ranges includes ranges which combine the values of the boundaries of all sub-ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1). Similarly, a range of 5 to 22, for example, includes the integers 5, 6, 7, 8, 9, 10, 11, 12, 13, 14, 15, 16, 17, 18, 19, 20, 21, and 22.

As used in this specification, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

Where used herein, the pronoun “we” is intended to refer to all persons involved in a particular aspect of the investigation disclosed herein and as such may include non-inventor laboratory assistants and non-inventor collaborators working under the supervision of the inventor(s).

Reference to “a” moiety, chemical, or chemical compound is intended to refer one or more (i.e., a plurality of) atoms or molecules of the substance and is not intended to be limited to a single atom or molecule of the substance unless explicitly indicated as referring to only a single atom or molecule of the substance. Furthermore, the plurality of atoms or molecules may or may not be identical, as long as they possess the same chemical formula or fall under the same category of chemical compound. For example, the plurality of atoms or molecules may comprise the same or different isotopes, isomers, enantiomers, or stereoisomers, or ratios thereof. Further, reference to a particular chemical class, such as a polymer or polyester, is intended to include one or more polymer or polyester molecules, and when in reference to a plurality of the molecules, the molecules making up the plurality may or may not be identical, and may differ, for example, in terms of different numbers of repeating units or having different molecular weights, as long as the molecules fall within a particular average or range of the repeating units or molecular weights.

Where used herein, the terms “specifically binds to,” “specific binding,” “binds specifically to,” and “binding specificity” refer to the ability of a ligand (e.g., an annexin) or other agent to detectably bind to a receptor or a binding epitope while having relatively little detectable reactivity with other proteins, epitopes, or receptor structures presented on cells to which the ligand or other agent may be exposed.

As used herein, a “protein-drug conjugate” refers to a molecule that contains at least one protein, such as an annexin, and at least one therapeutic moiety such as a drug that is covalently linked to the protein. They may be coupled directly or via a linker and in certain embodiments may be produced by chemical coupling methods or by recombinant expression of chimeric DNA molecules to produce fusion proteins.

As used herein, the terms “covalently coupled,” “linked,” “operably-linked,” “bonded,” “joined,” and the like, with reference to the protein and the drug components of the conjugates of the present disclosure, mean that the specified components are either directly covalently bonded to one another or indirectly covalently bonded to one another through an intervening moiety or components, such as (but not limited to) a bridge, spacer, linker or the like. Operably-linked moieties are associated in such a way so that the function of one moiety is not affected by the other, i.e., the moieties are connected in such an arrangement that they are configured so as to perform their usual function. The two moieties may be linked directly, or may be linked indirectly via a linker sequence of molecule. A non-limiting example of a linkage is the covalent linking of the protein and the drug by a flexible oligopeptide linker.

Where used herein, the terms “annexin” or “annexin protein” refers to any of annexins 1-11 and 13, which are more particularly designated as annexins A1, A2, A3, A4, A5, A6, A7, A8, A9, A10, A11, and A13. Annexin I and annexin V, where used herein, refer to Annexin A1 and Annexin A5, respectively, for example. The annexins contemplated for use herein include both human annexins and non-human cognate orthologs of annexins A1-A11 and A13 from non-human vertebrates, including but not limited to, non-human primates, dogs, cats, horses, livestock animals and zoo animals, which may be used for treatment in said non-human mammals in the methods contemplated herein. The annexins contemplated for use herein are discussed in further detail in V. Gerke and S.E. Moss (Physiol. Rev., (2002) 82:331-371). The terms “annexin” or “annexin protein” as used herein may also include fragments of annexins, instead of an entire annexin, as long as the fragment retains the PS binding activity of an entire annexin. In particular, Annexin A5 binds with very high affinity to PS-containing phospholipid bilayers. Annexin 5 may be obtained, for example, as described in U.S. Pat. No. 7,393,833, issued to Lind et al. on Jul. 1, 2008.

The term “effective amount” refers to an amount of the conjugate sufficient to exhibit a detectable therapeutic effect when used in the manner of the present disclosure. The therapeutic effect may include, for example but not by way of limitation, reducing the viability or numbers of TNBC cells in a subject, or extending the survival of the subject, or ameliorating the symptoms of a disease in the subject. The effective amount for a subject will depend upon the type of subject, the subject's size and health, the nature and severity of the condition to be treated, the method of administration, the duration of treatment, the nature of concurrent therapy (if any), the specific formulations employed, and the like. The effective amount for a given situation can be determined by one of ordinary skill in the art using routine experimentation based on the information provided herein.

The term “ameliorate” means a detectable or measurable improvement in a subject's condition or symptom thereof. A detectable or measurable improvement includes a subjective or objective decrease, reduction, inhibition, suppression, limit or control in the occurrence, frequency, severity, progression, or duration of the condition, or symptoms associated therewith, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject.

A decrease or reduction in worsening, such as stabilizing the condition or disease, e.g., TNBC, is also a successful treatment outcome. A therapeutic benefit therefore need not be complete ablation or reversal of the TNBC, or any single, most, or all adverse symptoms, complications, consequences or underlying causes associated with the disease or condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the TNBC (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

As used herein, the term “concurrent therapy” is used interchangeably with the terms “combination therapy” and “adjunct therapy,” and will be understood to mean that the patient in need of treatment is treated or given another drug for the disease in conjunction with the conjugates of the present disclosure. This concurrent therapy can be sequential therapy where the patient is treated first with one drug and then the other, or the two drugs are given simultaneously.

The term “pharmaceutically acceptable” refers to compounds and compositions which are suitable for administration to humans and/or animals without undue adverse side effects.

The term “active agent” where used herein is intended to refer to a substance which possesses a biological activity relevant to the present disclosure, and particularly refers to therapeutic and diagnostic substances which may be used in methods described in the present disclosure. By “biologically active” is meant the ability to modify the physiological system of a cell, tissue, or organism without reference to how the active agent has its physiological effects. Where used herein, unless otherwise noted, the term “active agent” includes pharmaceutically-acceptable salts, hydrates, solvates, and amorphous solids thereof.

As used herein, “substantially pure” means an object species is the predominant species present (i.e., on a molar basis it is more abundant than any other individual species in the composition). In certain embodiments, a substantially purified fraction is a composition wherein the object species comprises at least about 50 percent (on a molar basis) of all macromolecular species present. In certain embodiments, a substantially pure composition will comprise more than about 80 percent of all macromolecular species present in the composition, or more than about 85%, or more than about 90%, or more than about 95%, or more than about 99% of all macromolecular species present in the composition.

A “liposome” is a small vesicle composed of various types of lipids, phospholipids and/or surfactant. The components of the liposome are commonly arranged in a bilayer formation, similar to the lipid arrangement of biological membranes.

The term “subject” is used interchangeably herein with the term “patient” and includes human and veterinary subjects. For purposes of treatment, the term “mammal” as used herein refers to any animal classified as a mammal, including (but not limited to) humans, non-human primates, monkeys, domestic animals (such as, but not limited to, dogs and cats), experimental mammals (such as mice, rats, rabbits, guinea pigs, and chinchillas), farm animals (such as, but not limited to, horses, pigs, cattle, goats, sheep, and llamas), and any other animal that has mammary tissue.

The terms “treat,” “treating,” and “treatment,” as used herein, will be understood to include both inhibition of cancerous cell growth or bacterial or parasite growth as well as killing parasites and/or infected cells.

The term “receptor” as used herein will be understood to include any peptide, protein, glycoprotein, lipoprotein, polycarbohydrate, or lipid that is expressed or overexpressed on the surface of a cell.

The term “homologous” or “% identity” as used herein means a nucleic acid (or fragment thereof) or a protein (or a fragment thereof) having a degree of homology to the corresponding natural reference nucleic acid or protein that may be in excess of 70%, or in excess of 80%, or in excess of 85%, or in excess of 90%, or in excess of 91%, or in excess of 92%, or in excess of 93%, or in excess of 94%, or in excess of 95%, or in excess of 96%, or in excess of 97%, or in excess of 98%, or in excess of 99%. For example, in regard to peptides or polypeptides, the percentage of homology or identity as described herein is typically calculated as the percentage of amino acid residues found in the smaller of the two sequences which align with identical amino acid residues in the sequence being compared, when four gaps in a length of 100 amino acids may be introduced to assist in that alignment (as set forth by Dayhoff, in Atlas of Protein Sequence and Structure, Vol. 5, p. 124, National Biochemical Research Foundation, Washington, D.C. (1972)). In one embodiment, the percentage homology as described above is calculated as the percentage of the components found in the smaller of the two sequences that may also be found in the larger of the two sequences (with the introduction of gaps), with a component being defined as a sequence of four, contiguous amino acids. Also included as substantially homologous is any protein product which may be isolated by virtue of cross-reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

In certain non-limiting embodiments, the dosage of the protein-drug conjugate administered to a subject could be in a range of 1 μg per kg of subject body mass to 1000 mg/kg, or in a range of 5 μg per kg to 500 mg/kg, or in a range of 10 μg per kg to 300 mg/kg, or in a range of 25 μg per kg to 250 mg/kg, or in a range of 50 μg per kg to 250 mg/kg, or in a range of 75 μg per kg to 250 mg/kg, or in a range of 100 μg per kg to 250 mg/kg, or in a range of 200 μg per kg to 250 mg/kg, or in a range of 300 μg per kg to 250 mg/kg, or in a range of 400 μg per kg to 250 mg/kg, or in a range of 500 μg per kg to 250 mg/kg, or in a range of 600 μg per kg to 250 mg/kg, or in a range of 700 μg per kg to 250 mg/kg, or in a range of 800 μg per kg to 250 mg/kg, or in a range of 900 μg per kg to 250 mg/kg, or in a range of 1 mg per kg to 200 mg/kg, or in a range of 1 mg per kg to 150 mg/kg, or in a range of 2 mg per kg to 100 mg/kg, or in a range of 5 mg per kg to 100 mg/kg, or in a range of 10 mg compound per kg to 100 mg/kg, or in a range of 25 mg per kg to 75 mg/kg. For example, in certain non-limiting embodiments, the composition could contain protein-drug conjugate in a range of 0.1 mg/kg to 10 mg/kg, or any range comprising a combination of said ratio endpoints, such as, for example, a range of 10 μg/kg to 10 mg/kg.

The dosage of an administered active agent for humans will vary depending upon factors such as (but not limited to) the patient's age, weight, height, sex, general medical condition, and previous medical history. In certain non-limiting embodiments, where the active agent is administered by injection or infusion, the recipient may be provided with a dosage of the active agent that is in the range of from about 1 mg to about 1000 mg, and it may be administered as a single infusion or multiple injections, although a lower or higher dosage also may be administered. In certain non-limiting embodiments, the dosage may be in the range of from about 25 mg to about 100 mg of the active agent per square meter (m2) of body surface area for a typical adult, although a lower or higher dosage also may be administered. Non-limiting examples of dosages of the active agent that may be administered to a human subject include, but are not limited to, those in ranges of 1 mg to 1000 mg, 1 mg to 600 mg, 1 mg to 500 mg, 1 mg to 400 mg, 1 mg to 300 mg, 1 mg to 200 mg, 100 mg to 600 mg, 100 mg to 500 mg, 100 mg to 400 mg, 100 mg to 300 mg, 100 mg to 200 mg, 150 mg to 600 mg, 150 mg to 500 mg, 150 mg to 400 mg, 150 mg to 300 mg, 150 mg to 250 mg, 150 mg to 200 mg, 200 mg to 7500 mg, 200 mg to 600 mg, 200 mg to 500 mg, 200 mg to 400 mg, 200 mg to 300 mg, and 200 mg to 250 mg, or any subrange within any of the aforementioned ranges. Dosages may be repeated as needed, for example (but not by way of limitation), once per week for 4 to 10 weeks, once per week for 8 weeks, or once per week for 4 weeks. It may also be given less frequently, such as (but not limited to) every other week for several months, or more frequently, such as twice weekly or by continuous infusion.

In certain non-limiting embodiments, the present disclosure is directed to a dosing regimen comprising multiple dosing cycles (e.g., wherein the first dosing cycle is a step-up, fractionated dosing cycle). In some non-limiting embodiments, the dose may range from 50 mg to 200 mg (e.g., from 50 mg to 175 mg, from 50 mg to 150 mg, from 50 mg to 125 mg, from 50 mg to 100 mg, from 50 mg to 75 mg, from 50 mg to 70 mg, from 52 mg to 100 mg, from 52 mg to 75 mg, from 50 mg to 180 mg, from 55 mg to 150 mg, from 55 mg to 100 mg, from 55 mg to 70 mg, from 55 mg to 65 mg, from 58 mg to 62 mg; e.g., about 60 mg, or any subrange within any of the aforementioned ranges). In some non-limiting embodiments, the dose may be about 60 mg. In some non-limiting embodiments, the dose is about 1 mg. In some non-limiting embodiments, the dose is about 2 mg.

In some non-limiting embodiments, the dose is from 20 mg to 200 mg (e.g., from 20 mg to 175 mg, from 20 mg to 150 mg, from 20 mg to 100 mg, from 20 mg to 75 mg, from 30 mg to 175 mg, from 40 mg to 175 mg, from 45 mg to 175 mg, from 50 mg to 175 mg, from 30 mg to 150 mg, from 40 mg to 100 mg, from 45 mg to 75 mg, from 50 mg to 70 mg, from 55 mg to 65 mg, from 58 mg to 62 mg; about 20 mg, about 30 mg, about 45 mg, or e.g., about 60 mg, or any subrange within any of the aforementioned ranges). In some non-limiting embodiments, the dose is from about 12 mg to about 48 mg (e.g., from about 12 mg to about 42 mg, from about 12 mg to about 36 mg, from about 12 mg to about 30 mg, from about 18 mg to about 48 mg, from about 18 mg to about 42 mg, from about 24 mg to about 42 mg, from about 27 mg to about 42 mg, from about 24 mg to about 36 mg, from about 27 mg to about 33 mg, from about 28 mg to about 32 mg; e.g., about 24 mg, about 27 mg, about 30 mg, about 33 mg, or about 36 mg, or any subrange within any of the aforementioned ranges).

In some non-limiting embodiments, the dosing regimen comprises administration of a dose in a range of from 100 mg to 750 mg (e.g., from 100 mg to 725 mg, from 100 mg to 700 mg, from 100 mg to 675 mg, from 100 mg to 650 mg, from 100 mg to 625 mg, from 100 mg to 600 mg, from 100 mg to 575 mg, from 100 mg to 550 mg, from 100 mg to 525 mg, from 100 mg to 500 mg, from 100 mg to 475 mg, from 100 mg to 450 mg, from 100 mg to 425 mg, from 100 mg to 400 mg, from 100 mg to 375 mg, from 100 mg to 350 mg, from 100 mg to 325 mg, from 100 mg to 300 mg, from 100 mg to 275 mg, from 100 mg to 250 mg, or from 100 mg to 225 mg, from 100 mg to 200 mg, from 100 mg to 175 mg, from 100 mg to 150 mg, or from 100 mg to 125 mg, or any subrange within any of the aforementioned ranges).

In some non-limiting embodiments, the dosing regimen comprises administration of a dose in a range of from 200 mg to 750 mg (e.g., from 200 mg to 725 mg, from 200 mg to 700 mg, from 200 mg to 675 mg, from 200 mg to 650 mg, from 200 mg to 625 mg, from 200 mg to 600 mg, from 200 mg to 575 mg, from 200 mg to 550 mg, from 200 mg to 525 mg, from 200 mg to 500 mg, from 200 mg to 475 mg, from 200 mg to 450 mg, from 200 mg to 425 mg, from 200 mg to 400 mg, from 200 mg to 375 mg, from 200 mg to 350 mg, from 200 mg to 325 mg, from 200 mg to 300 mg, from 200 mg to 275 mg, from 200 mg to 250 mg, or from 200 mg to 225 mg, or any subrange within any of the aforementioned ranges).

In some non-limiting embodiments, the dosing regimen comprises administration of a dose in a range of from 300 mg to 750 mg (e.g., from 300 mg to 725 mg, from 300 mg to 700 mg, from 300 mg to 675 mg, from 300 mg to 650 mg, from 300 mg to 625 mg, from 300 mg to 600 mg, from 300 mg to 575 mg, from 300 mg to 550 mg, from 300 mg to 525 mg, from 300 mg to 500 mg, from 300 mg to 475 mg, from 300 mg to 450 mg, from 300 mg to 425 mg, from 300 mg to 400 mg, from 300 mg to 375 mg, from 300 mg to 350 mg, or from 300 mg to 325 mg, or any subrange within any of the aforementioned ranges).

In some non-limiting embodiments, the dosing regimen comprises administration of a dose in a range of from 400 mg to 750 mg (e.g., from 400 mg to 725 mg, from 400 mg to 700 mg, from 400 mg to 675 mg, from 400 mg to 650 mg, from 400 mg to 625 mg, from 400 mg to 600 mg, from 400 mg to 575 mg, from 400 mg to 550 mg, from 400 mg to 525 mg, from 400 mg to 500 mg, from 400 mg to 475 mg, from 400 mg to 450 mg, or from 400 mg to 425 mg, or any subrange within any of the aforementioned ranges).

In some non-limiting embodiments, the dosing regimen comprises administration of a dose in a range of from 500 mg to 750 mg (e.g., from 500 mg to 725 mg, from 500 mg to 700 mg, from 500 mg to 675 mg, from 500 mg to 650 mg, from 500 mg to 625 mg, from 500 mg to 600 mg, from 500 mg to 575 mg, from 500 mg to 550 mg, or from 500 mg to 525 mg, or any subrange within any of the aforementioned ranges).

In some non-limiting embodiments, the dosing regimen comprises administration of a dose in a range of from 600 mg to 750 mg (e.g., from 600 mg to 725 mg, from 600 mg to 700 mg, from 600 mg to 675 mg, from 600 mg to 650 mg, from 600 mg to 625 mg, or any subrange within any of the aforementioned ranges).

In some non-limiting embodiments, the active agent is provided in a concentration of about 1 nM, about 5 nM, about 10 nM, about 25 nM, about 50 nM, about 75 nM, about 100 nM, about 150 nM, about 200 nM, about 250 nM, about 300 nM, about 350 nM, about 400 nM, about 500 nM, about 550 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 60 μM, about 70 μM, about 75 μM, about 80 μM, about 90 μM, about 100 μM, about 125 μM, about 150 μM, about 175 μM, about 200 μM, about 250μM, about 300 μM, about 350 μM, about 400 μM, about 500 μM, about 600 μM, about 700 μM, about 750 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 400 mM, about 500 mM, about 600 mM, about 700 mM, about 800 mM, about 900 mM, about 1000 mM, about 1 M, about 1.1 M, about 1.2 M, about 1.3 M, about 1.4 M, about 1.5 M, about 1.6 M, about 1.7 M, about 1.8 M, about 1.9 M, about 2 M, about 3 M, about 4 M, about 5 M, about 6 M, about 7 M, about 8 M, about 9 M, about 10 M, about 15 M, about 20 M, about 25 M, about 30 M, about 35 M, about 40 M, about 45 M, about 50 M, about 75 M, about 100 M, or any range in between any two of the aforementioned concentrations, including said two concentrations as endpoints of the range, or any number in between any two of the aforementioned concentrations.

When administered orally, the active agent composition may be protected from digestion. This can be accomplished either by complexing the active agent with a composition to render it resistant to acidic and enzymatic hydrolysis or by packaging the active agent in an appropriately resistant carrier such as (but not limited to) a liposome, e.g., such as shown in U.S. Pat. No. 5,391,377.

The active agents of the present disclosure can be administered to a subject by any of a number of effective routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compounds may also be formulated for inhalation. In certain embodiments, a compound may be simply dissolved or suspended in sterile water. Oral formulations may be formulated such that the active agents pass through a portion of the digestive system before being released, for example it may not be released until reaching the small intestine, or the colon. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

Tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations which are suitable for vaginal administration also include pessaries, tampons, creams, gels, pastes, foams or spray formulations containing such carriers as are known in the art to be appropriate. In certain embodiments, the active agents of the present disclosure can be formulated into suppositories, slow-release formulations, or intrauterine delivery devices (IUDs).

Formulations of the pharmaceutical compositions for administration to the mouth may be presented as a mouthwash, or an oral spray, or an oral ointment.

Alternatively, or additionally, compositions can be formulated for delivery via a catheter, stent, wire, or other intraluminal device. Delivery via such devices may be especially useful for delivery to the bladder, urethra, ureter, rectum, or intestine.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required. The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection and infusion.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical transdermal administration, the agents are formulated into ointments, creams, salves, powders, and gels. Transdermal delivery systems can also include (for example but not by way of limitation) patches. The present compositions can also be administered in sustained delivery or sustained release mechanisms. For example, biodegradable microspheres or capsules or other biodegradable polymer configurations capable of sustained delivery can be included herein.

The compositions of the present disclosure may be formulated as implants, in the form of either biodegradable microparticles or small squared films comprising the microparticles of the present disclosure. The following describes methods of making such implants. Microparticles (e.g., 5-100 micrometers) containing different loadings of the conjugates of the present disclosure, may be prepared by spray drying suspensions of the nanocrystals of the compounds and a biodegradable polymer (for example, polylactic acid of molecular weights 50,000-100,000) or polylactic-co-glycolic acid copolymer (e.g., proportions 75:25 or 50:50). Microparticles may contain, e.g., 10-50% wt/wt drug: polymer and can be implanted alone, or in a biodegradable film, e.g., as an implantable chitosan-egg phosphatidylcholine (ePC) films. To make such chitosan-ePC films, chitosan flakes and ePC can be dissolved in a 1% acetic acid at a ratio of 1:0.8 (wt/wt). Microparticles containing the drug in nanocrystal form are dispersed in the chitosan-ePC solution in different proportions (e.g., 1:3, 1:5, 1:7, and 1:10 wt/wt) to achieve the release of different drug doses. The resulting microparticle-chitosan-ePC suspension can be poured into a Teflon dish to have a 2-3 mm thickness and allowed to dry in a covered dessicator for 5 days. After the films are dry, they can be cut into small squares of 15×15 mm². The implants can be made in different forms, including but not limited to thin films, rods, and wafers. Other biodegradable polymers that can be used to make implants include, but are not limited to, poly-lactic acid, poly-lactic-co-glycolic acid copolymer, poly-caprolactone, poly-sebacic acid, poly-adipic acid, poly 3(3-hydroxybutyric acid), poly(3-hydroxybutyrate-co-3-hydroxyvalerate, poly-trimethylene carbonate, chitosan, chitin, gelatin, collagen, and hyaluronic acid.

In non-limiting embodiments, gels comprising the active agents of the present disclosure can be made by combining the active agents in various proportions to a sodium alginate gel base or carbomer jelly base to form a homogeneous gel suitable for topical application.

In non-limiting embodiments, ointments comprising the active agents of the present disclosure can be made by combining the active agents in various proportions to a Hydrophilic Petrolatum USP base, Lanolin, USP base or to Polyethylene glycol ointment, NF to form a homogeneous ointment suitable for topical application.

In non-limiting embodiments, creams comprising the active agents of the present disclosure can be made by combining the active agents in various proportions in suspension in water and glycerin (e.g., 20:1 parts) and emulsified in a mixture of e.g., Lanolin, Beeswax USP-NF and Cetyl alcohol, plus a Tween 80 and Span 80.

Several gel, ointment, and/or cream compositions that can be used are shown in Garcia-Contreras L, Abu-Izza K, Lu DR. “Biodegradable cisplatin microspheres for direct brain injection: Preparation and characterization.” Pharm Dev Technol (1997)2(1): 53-65.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another non-limiting aspect, the device for delivering the formulation to respiratory tissue is an inhaler in which the formulation vaporizes. Other liquid delivery systems include (for example but not by way of limitation) air jet nebulizers.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another non-limiting aspect, the device for delivering the formulation to respiratory tissue is an inhaler in which the formulation vaporizes. Other liquid delivery systems include (for example, but not by way of limitation) air jet nebulizers.

Pharmaceutical compositions suitable for parenteral administration comprise one or more active compounds in combination with one or more pharmaceutically acceptable sterile isotonic aqueous or nonaqueous solutions, dispersions, suspensions, or emulsions, or sterile powders which may be reconstituted into sterile injectable solutions or dispersions just prior to use, which may contain antioxidants, buffers, bacteriostats, solutes which render the formulation isotonic with the blood of the intended recipient, or suspending or thickening agents.

Examples of suitable aqueous and nonaqueous carriers that may be employed in the pharmaceutical compositions of the present disclosure include (but are not limited to) water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot injectable formulations are also prepared by entrapping the drug in liposomes or microemulsions that are compatible with body tissue.

As noted, effective amounts of the active agents may be administered orally, in the form of a solid or liquid preparations such as capsules, pills, tablets, lozenges, melts, powders, suspensions, solutions, elixirs or emulsions. Solid unit dosage forms can be capsules of the ordinary gelatin type containing, for example, surfactants, lubricants, and inert fillers such as lactose, sucrose, and cornstarch, or the dosage forms can be sustained release preparations. The pharmaceutical composition may contain a solid carrier, such as a gelatin or an adjuvant. The tablet, capsule, and powder may contain from about 0.05 to about 95% of the active substance compound by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition particularly contains from about 0.005 to about 95% by weight of the active agent(s). For example, a dose of about 10 mg to about 1000 mg once or twice a day could be administered orally.

In another embodiment, the active agents of the present disclosure can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the active agents in an aqueous or non-aqueous pharmaceutically acceptable solvent which may also contain suspending agents, sweetening agents, flavoring agents, and preservative agents as are known in the art.

In one non-limiting aspect, the active agent is incorporated in lipid monolayers or bilayers, such as (but not limited to) liposomes. Liposomes and liposomal formulations can be prepared according to standard methods and are also well known in the art, such as (but not limited to) those disclosed in U.S. Pat. Nos. 6,110,490; 6,096,716; 5,283,185; 5,279,833; 4,235,871; 4,501,728; and 4,837,028.

In one non-limiting aspect, the compositions are prepared with carriers that will protect the active agent against rapid elimination from the body, such as (but not limited to) a controlled release formulation, including implants and microencapsulated delivery systems. Biodegradable, biocompatible polymers can be used, such as (but not limited to) ethylene vinyl acetate, polyanhydrides, polyglycolic acid, collagen, polyorthoesters, and polylactic acid. Methods for preparation of such formulations will be apparent to those skilled in the art.

The active agents in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active agent is combined in a mixture with one or more pharmaceutically suitable excipients. Sterile phosphate-buffered saline is one non-limiting example of a pharmaceutically suitable excipient.

The acid addition salts may include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecyl sulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N?-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and di-n-ate.

Non-limiting examples of routes of administration of the compositions described herein include parenteral injection, e.g., by subcutaneous, intramuscular, or transdermal delivery. Other forms of injection include (but are not limited to) intravenous, intraarterial, intralymphatic, intrathecal, intraocular, intranasal, intracranial, intracerebral, intraperitoneal, or intracavitary injection. In parenteral administration, the compositions will be formulated in a unit dosage injectable form such as (but not limited to) a solution, suspension, or emulsion, in association with a pharmaceutically acceptable excipient. Such excipients are inherently nontoxic and nontherapeutic. Non-limiting examples of such excipients include saline, Ringer's solution, dextrose solution, and Hanks' solution. Nonaqueous excipients such as (but not limited to) fixed oils and ethyl oleate may also be used. An alternative non-limiting excipient is 5% dextrose in saline. The excipient may contain minor amounts of additives such as (but not limited to) substances that enhance isotonicity and chemical stability, including buffers and preservatives. The active agents can be delivered or administered alone or as pharmaceutical compositions by any means known in the art, such as (but not limited to) systemically, regionally, or locally, for example by intraarterial, intrathecal (IT), intravenous (IV), parenteral, intrapleural cavity, topical, oral, or local administration, as subcutaneous, intratracheal (e.g., by aerosol) or transmucosal administration (e.g., buccal, bladder, vaginal, uterine, rectal, and/or nasal mucosa). Administration can also be localized directly into a tumor. Administration into the systemic circulation by intravenous, inhalation, mucosal, or subcutaneous administration is typical. Intravenous administration can be, for example (but not by way of limitation), by infusion over a period such as (but not limited to) 30-90 min or by a single bolus injection, or by other regimens as described elsewhere herein.

For parenteral administration, for example, the active agents may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives and buffers as are known in the art.

When an effective amount of the active agents is administered by intravenous, cutaneous, or subcutaneous injection, the compound is particularly in the form of a pyrogen-free, parenterally acceptable aqueous solution or suspension. The preparation of such parenterally acceptable solutions, having due regard to pH, isotonicity, stability, and the like, is well within the skill in the art. A particular pharmaceutical composition for intravenous, cutaneous, or subcutaneous injection may contain, in addition to the active agent, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical compositions of the present disclosure may also contain stabilizers, preservatives, buffers, antioxidants, or other additives known to those of skill in the art.

As noted, particular amounts and modes of administration can be determined by one skilled in the art. One skilled in the art of preparing formulations can readily select the proper form and mode of administration, depending upon the particular characteristics of the active agents selected, the condition to be treated, the stage of the condition, and other relevant circumstances using formulation technology known in the art, described, for example, in Remington: The Science and Practice of Pharmacy, 22nd ed.

Additional pharmaceutical methods may be employed to control the duration of action of the active agents. Increased half-life and/or controlled release preparations may be achieved through the use of proteins or polymers to conjugate, complex with, and/or absorb the active agents as discussed previously herein. The controlled delivery and/or increased half-life may be achieved by selecting appropriate macromolecules (for example but not by way of limitation, polysaccharides, polyesters, polyamino acids, homopolymers, polyvinyl pyrrolidone, ethylenevinylacetate, methylcellulose, or carboxymethylcellulose, and acrylamides such as N-(2-hydroxypropyl)methacrylamide), and the appropriate concentration of macromolecules as well as the methods of incorporation, in order to control release.

Another possible method useful in controlling the duration of action of the active agents by controlled release preparations and half-life is incorporation of the active agents or their functional derivatives into particles of a polymeric material such as polyesters, polyamides, polyamino acids, hydrogels, poly(lactic acid), ethylene vinylacetate copolymers, copolymer micelles of, for example, PEG and poly(l-aspartamide). Additional pharmaceutical methods may be employed to increase bioavailability of the drug, such as Kolliphor HS15.

It is also possible to entrap the active agents in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nanoparticles, and nanocapsules), or in macroemulsions. Such techniques are well known to persons having ordinary skill in the art.

When the active agents are to be used as an injectable material, they can be formulated into a conventional injectable carrier. Suitable carriers include biocompatible and pharmaceutically acceptable phosphate buffered saline solutions, which are particularly isotonic.

For reconstitution of a lyophilized product in accordance with the present disclosure, one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field. The pharmaceutical composition may also be in the form of an aqueous solution containing many of the same substances as described above for the reconstitution of a lyophilized product.

The active agents can also be administered as a pharmaceutically acceptable acid-or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, tauric acid, maleic acid, and fumaric acid, or by reaction with an inorganic base such as sodium hydroxide, ammonium hydroxide, potassium hydroxide, and organic bases such as monoalkyl, dialkyl, trialkyl and aryl amines, and substituted ethanolamines.

In certain embodiments, the present disclosure includes an active agent composition wherein at least one of the active agents is coupled (e.g., by covalent bond) directly or indirectly to a carrier molecule.

Formulated compositions comprising the active agents of the present disclosure can be provided in unit dosage form, e.g., in ampoules or in multi-dose containers, with an added preservative. Compositions can also take such forms as suspensions, solutions, or emulsions in oily or aqueous vehicles, and can contain formulatory agents such as suspending, stabilizing, and/or dispersing agents.

In some non-limiting methods, the patient is administered the active agent every one, two, three, or four weeks, for example. The dosage depends on the frequency of administration, condition of the patient, response to prior treatment (if any), whether the treatment is prophylactic or therapeutic, and whether the disorder is acute or chronic, among other factors.

The number of dosages administered may depends on the severity and temporal nature of the disorder (e.g., whether presenting acute or chronic symptoms) and the response of the disorder to the treatment. For acute disorders or acute exacerbations of a chronic disorder, between 1 and 10 doses may be used. Sometimes a single bolus dose, optionally in divided form, is sufficient for an acute disorder or acute exacerbation of a chronic disorder. Treatment can be repeated for recurrence of an acute disorder or acute exacerbation. For chronic disorders, the active agent may be administered at regular intervals, such as (but not limited to) weekly, fortnightly, monthly, quarterly, every six months for at least 1, 5, or 10 years, or for the life of the patient.

Compositions can be administered in a single dose treatment or in multiple dose treatments on a schedule and over a time period appropriate to the age, weight, and condition of the subject, the particular composition used, and the route of administration. In one non-limiting embodiment, a single dose of the composition according to the disclosure is administered. In other non-limiting embodiments, multiple doses are administered. The frequency of administration can vary depending on any of a variety of factors, e.g., severity of the symptoms, degree of immunoprotection desired, or whether the composition is used for prophylactic or curative purposes. For example, in certain non-limiting embodiments, the composition is administered once per month, twice per month, three times per month, every other week, once per week, twice per week, three times per week, four times per week, five times per week, six times per week, every other day, daily, twice a day, or three times a day. The duration of treatment (i.e., the period of time over which the composition is administered) can vary, depending on any of a variety of factors, e.g., subject response. For example (but not by way of limitation), the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

The dosage of an administered active agent for humans will vary depending upon factors such as (but not limited to) the patient's age, weight, height, sex, general medical condition, and previous medical history. A dosage may be provided as several smaller amounts. For example, a single dosage of 500 mg may be administered as ten 50 mg tablets or capsules, or as five 100 mg tablets or capsules. The amounts of doses or dosages described herein may be provided in a single capsule, tablet, injection, infusion, or other more of delivery. Or, the amounts of drug which comprise the doses or dosages described herein may be provided in two or more (e.g., 2, 3, 4, 5, 6, 7, 8, 9, 10, 11, or 12) capsules, tablets, injections, infusions, or other modes of delivery.

In certain non-limiting embodiments, the recipient may be provided with a dosage of the active agent that is in the range of from about 1 mg to about 1000 mg. A lower or higher dosage also may be administered. In certain non-limiting embodiments, the dosage may be in the range of from about 25 mg to about 100 mg of the active agent per square meter (m²) of body surface area for a typical adult, although a lower or higher dosage also may be administered. Dosages may be repeated as needed, for example (but not by way of limitation), once per week for 4-10 weeks, once per week for 8 weeks, or once per week for 4 weeks. In certain non-limiting embodiments, the dosage can be provided as an infusion, for example as a single injection or as multiple injections. It may also be given less frequently, such as (but not limited to) every other week for several months, or more frequently, such as twice weekly, or by continuous infusion.

While the compositions and methods of the present disclosure have been described in terms of particular embodiments, it will be apparent to those of ordinary skill in the art that variations may be applied to the compositions and/or methods and in the steps or in the sequence of steps of the methods described herein without departing from the concept, spirit and scope of the inventive concepts. All such similar substitutes and modifications apparent to those skilled in the art are deemed to be within the spirit, scope, and concept of the inventive concepts as described herein.

In certain embodiments, the drug has a single carboxylic or a thiol functional group as a linking moiety. If the drug does not have a carboxylic or thiol functional group, a primary amine can be used as the drug's linking moiety.

In certain embodiments, the drug can be attached to annexin with linkers such as carbodiimide or hydroxymethyl phosphine and linker esters such as imidoester, NHS-ester, and pentafluorophenyl esters.

The chemical modification of chemotherapeutic functional groups that directly participate in antibacterial activity can reduce or eliminate the antibacterial activity of the protein-drug conjugate. For instance, the reaction schema should avoid conjugation chemistry that inactivates the antibiotic by damaging pharmacologically activate functional groups. For example, when linking members of the aminoglycoside class to annexin with linkers of the hydrazide class of linkers. This synthesis first requires oxidation of antibiotic sugar glycols using sodium periodate. During this reaction, the ring opening of vicinal diols by sodium periodate damages the aminoglycoside's saccharide rings of the aminoglycoside and reduces the antimicrobial activity of the resulting conjugate. In contrast to the use of hydrazine linkers where the protein can be inactivated by breaking the chemotherapeutics' antibacterial moieties, other linkers can render the conjugate less active by adding moieties that block the antibacterial active site of the antibiotic. For example, the linking process during the conjugation of cephalosporins to annexin with linkers such as the photoreactive diazirine family can sterically block the beta-lactam ring, preventing its reaction with the bacterial enzymes.

Linkers that work with this chemistry may be built of two or more crosslinking moieties that react with different target groups. Many crosslinking agents that fit this description are called click chemistry reagents. Crosslinking moieties that are compatible with this chemistry include carbodiimides, NHS esters, pentafluorophenyl esters, hydroxymethyl phosphines, maleimides, haloacetyls, pyridyldisulfides, thiosulfinates, and vinylsulfones. These crosslinking moieties can be combined together to produce complex linkers that connect functional groups.

Examples of cytotoxic drugs that can be used in the protein-drug conjugates of the present disclosure include, but are not limited to, in general, alkylating agents, anti-proliferative agents, tubulin binding agents and the like, for example, the anthracycline family of drugs, the vinca drugs, the mitomycins, the bleomycins, the cytotoxic nucleosides, the pteridine family of drugs, diynenes, and the podophyllotoxins. Examples of those groups include, adriamycin, carminomycin, daunorubicin, aminopterin, methotrexate, methopterin, dichloromethotrexate, mitomycin C, porfiromycin, 5-fluorouracil, 6-mercaptopurine, cytosine arabinoside, podophyllotoxin, or podophyllotoxin derivatives such as etoposide or etoposide phosphate, melphalan, vinblastine, vincristine, leurosidine, vindesine, leurosine, and the like. The drug may be selected from camptothecin, homocamptothecin, colchicine, combretastatin, dolistatin, doxorubicin, methotrexate, podophyllotixin, rhizoxin, rhizoxin D, a taxol, paclitaxol, CC1065, or a maytansinoid, and derivatives and analogs thereof.

The drugs of the conjugates of the present disclosure may be an antineoplastic agent such as Acivicin; Aclarubicin; Acodazole Hydrochloride; Acronine; Adozelesin; Adriamycin; Aldesleukin; Altretamine; Ambomycin; A. metantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Camptothecin; Caracemide; Carbetimer; Carboplatin; Carmustine; Carubicin Hydrochloride; Carzelesin; Cedefingol; Chlorambucil; Cirolemycin; Cisplatin; Cladribine; Colchicine; Combretestatin A-4; Crisnatol Mesylate; Cyclophosphamide; Cytarabine; Dacarbazine; DACA (N-[2-(Dimethyl-amino) ethyl]acridine-4-carboxamide); Dactinomycin; Daunorubicin Hydrochloride; Daunomycin; Decitabine; Dexormaplatin; Dezaguanine; Dezaguanine Mesylate; Diaziquone; Docetaxel; Dolasatins; Doxorubicin; Doxorubicin Hydrochloride; Droloxifene; Droloxifene Citrate; Dromostanolone Propionate; Duazomycin; Edatrexate; Eflornithine Hydrochloride; Ellipticine; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; Gemcitabine; Gemcitabine Hydrochloride; Gold Au 198; Homocamptothecin; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Mertansine (DM1); Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; PeploycinSulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Rhizoxin; Rhizoxin D; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiocolchicine; Thiamiprine; Thioguanine; Thiotepa; Thymitaq; Tiazofurin; Tirapazamine; Tomudex; TOP53; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′ Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N, N′-Bis (2-chloroethyl)-N-nitrosourea (BCNU); N-(2-chloroethyl)-N′ cyclohexyl-N-nitrosourea (CCNU); N-(2-chloroethyl)-N′-(trans-4-methylcyclohexyl-N-nitrosourea (MeCCNU); N-(2-chloroethyl)-N′-(diethyl) ethylphosphonate-N-nitrosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin;C1-973; DWA 2114R; JM216; JM335; Bis (platinum); tomudex; azacitidine; cytarabine; gemcitabine; 6-Mercaptopurine; 6-Thioguanine; Hypoxanthine; teniposide 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans apthal; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); or 2-chlorodeoxyadenosine (2-Cda).

Other suitable anti-neoplastic compounds include, but are not limited to, 20-pi-1,25 dihydroxyvitamin D3; 5-ethynyluracil; abiraterone; aclarubicin; acylfulvene; adecypenol; adozelesin; aldesleukin; all-tyrosine kinase antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; argininedeaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; basic fibroblast growth factor (bFGF) inhibitor, bicalutamide; bisantrene; bisaziridinylspermine; bisnafide; bistratene A; bizelesin; breflate; bleomycin A2; bleomycin B2; bropirimine; budotitane; buthionine sulfoximine; calcipotriol; calphostin C; camptothecin derivatives (e. g., 10-hydroxy-camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; and cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; 2′deoxycoformycin (DCF); deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflornithine; elemene; emitefur; epirubicin; epothilones; epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; homoharringtonine (HHT); hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maytansine; mannostatin A; marimastat; masoprocol; maspin; matrilysin inhibitors; matrix metalloproteinase inhibitors; menogaril; merbarone; meterelin; methioninase; metoclopramide; MIF inhibitor; ifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues and derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; rapamycin; ras farnesyl protein transferase inhibitors; ras inhibitors; ras-GAP inhibitor, retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. The drug may be an antiproliferative agent, for example piritrexim isethionate, or an antiprostatic hypertrophy agent such as, for example, sitogluside, a benign prostatic hyperplasia therapy agent such as, for example, tamsulosin hydrochloride, or a prostate growth inhibitor such as, for example, pentomone.

In certain embodiments, the presently disclosed drug conjugates may be used in combination with an immunostimulant. The destruction of the tumor cells and/or tumor vasculature causes tumor antigens to be released into the bloodstream. Tumor antigens alone may not be sufficient to stimulate an appropriate immune response. However, the addition of an immunostimulant has been shown to significantly enhance the immune response of the host to the tumor cells, which allows the immune system to mount a systemic attack on the remaining cells of the tumor. Any immunostimulant known in the art or otherwise capable of functioning in accordance with the present disclosure may be utilized in the compositions, methods and kits described herein. Examples of immunostimulants that may be utilized herein include, but are not limited to, cyclophosphamide, glycated chitosan; muramyldipeptide derivatives; trehalose-dimycolates; and BCG-cell wall skeleton; various cytokines; and combinations and/or derivatives thereof. Dosages of immunostimulants can be in the range of, for example, 0.001 to 100 mg/kg of body weight/day, depending on the method of administration.

The methods described herein may thus include the step of administering an effective amount of an immunostimulant, wherein the immunostimulant is effective in significantly enhancing the immune response of the patient to the tumor cells, and thereby allowing the immune system to mount a systemic attack on the remaining cells of the tumor. The immunostimulant may be administered at the same time as either the drug conjugate, or may be administered before or after the administration of the drug conjugate. Alternatively, the immunostimulant may be administered multiple times to the patient.

In the same manner, the methods described herein may include the step of administering an effective amount of an mTOR inhibitor, wherein the mTOR inhibitor is effective in directly or indirectly decreasing the activity of TOR. The mTOR inhibitor may be administered at the same time as the drug conjugate or may be administered before or after the administration of the drug conjugate. Alternatively, the mTOR inhibitor may be administered multiple times to the patient. Examples of mTOR inhibitors include but are not limited to rapamycin (sirolimus), everolimus (RAD001), temsirolimus (CCI-779), ridaforolimus (deforolimus, AP-23573), metformin, tacrolimus, ABT-578, AP23675, AP-23841, 7-epi-rapamycin, 7-thiomethyl-rapamycin, 7-epi-tromethoxyphenyyl-rapamycin, 7-epi-thiomethyl-rapamycin, 7-demethoxy-rapamycin, 32-demethoxy-rapamycin, 7-desmethyl-rapamycin, 42-O-(2-hydroxy) ethyl-rapamycin, and other analogs of rapamycin (“rapalogs”).

One skilled in the art may make any suitable chemical modifications to the above compounds in order to make reactions of that compound more convenient for purposes of preparing the protein-drug conjugates.

### EXAMPLES

The inventive concepts of the present disclosure will now be discussed in terms of several specific, non-limiting, examples. The examples described below, which include particular embodiments, will serve to illustrate the practice of the present disclosure, it being understood that the particulars shown are by way of example and for purposes of illustrative discussion of particular embodiments of the present disclosure only and are presented in the cause of providing what is believed to be a useful and readily understood description of procedures as well as of the principles and conceptual aspects of the inventive concepts.

### METHODS

### Protein Production

Recombinant ANXA5 was produced in BL21 (DE3) E. coli transfected with a pET-30 Ek/LIC/ANXA5 plasmid. Bacteria were grown in Luria broth medium, and protein production was induced by IPTG. The resulting protein was purified using an N-terminal polyhistidine-tag for purification by IMAC with immobilized Ni²⁺ (GE Healthcare Life Sciences, Meadowvale, ON, Canada). An engineered HRV 3C protease cleavage site (LEVLFQ↓GP) between the polyhistidine tag and ANXA5 enabled this tag to be removed by HRV 3C protease (Thermo Fisher Scientific, Waltham, MA, USA). The ANXA5 gene sequence was verified by DNA sequencing at the Oklahoma Medical Research Foundation (Oklahoma City, OK). Recombinant protein was confirmed as greater than 95% purity by SDS-PAGE and endotoxin free by Limulus assay (Thermo Fisher Scientific, Waltham, MA, USA).

### Synthesis of annexin A5 mertansine (ANXA5-DM1) Conjugate

Mertansine (DM1-MedChemExpress, Monmouth Junction, NY, USA)) was linked to the primary amines (lysine residues) of ANXA5 with the heterobifunctional crosslinker sulfo-SMCC, (TCI, Portland, OR, USA). First, 13.8 mM (6 mg/ml) of sulfo-SMCC is dissolved in DI water. If the sulfo-SMCC does not fully dissolve, the sulfo-SMCC mixture is gently heated to 40-50° C. The solution is allowed to cool to room temperature before the addition of the protein. Next, 200 μL of the sulfo-SMCC solution is added to 1 ml of 27.77 μM (1 mg/ml) ANXA5 and allowed to react for 1 h at 4° C. on an orbital shaker. The maleimide moiety of the sulfo-SMCC linker readily captures the only thiol functional group in DM1. DM1 is dissolved in DMSO (Fisher Scientific, Pittsburgh, PA, USA) at a concentration of 9.03 mM (6.66 mg/ml). The DM1 mixture (150 μL) is added to ANXA5-sulfo-SMCC solution and allowed to react for 2 h at 4° C. on an orbital shaker. The resulting ANXA5-DM1 biconjugate is then purified from excess free DM1 with 8-h dialysis in 2 L of 30 mM sodium phosphate buffer at pH 7.4 with a regenerated cellulose dialysis membrane of MWCO 12-14 kDa (Fisher Scientific, Pittsburgh, PA, USA) at 4° C. Dialysate is changed at least once to ensure removal of unreacted molecules.

### Characterization of ANXA5-DM1

The ANXA5-DM1 bioconjugate was characterized by absorbance spectroscopy, SDS-PAGE gel electrophoresis, and denaturing mass analysis. In the spectroscopic assay, the concentration of DM1 in the ANXA5-DM1 conjugate was determined by measuring the absorbance at 288 nm (peak absorbance for DM1) and then subtracting the absorbance contributed by the protein at this wavelength and taking into account the protein concentration, which was determined by the Bradford protein assay. SDS-PAGE analysis was also used to validate drug loading. ANXA5-DM1 was denatured in 2× Laemelli sample buffer (Bio-Rad, Hercules, CA, USA) and heated to 100° C. for 5 min. Samples were transferred to 4-20% mini-protean TGX precast 10-well gradient gels (Bio-Rad, Hercules, CA, USA). The samples were run at 200 V for 30 minutes in 1% tris-glycine-SDS running buffer (Bio-Rad, Hercules, CA, USA), stained with Imperial stain (Thermo Fisher, Waltham, MA, USA), and destained with DI water overnight.

For the denaturing intact mass analysis (performed at the Department of Chemistry and Molecular Biology, University of Texas Medical Branch, Galveston, TX), samples containing purified ANXA5-DM1 (0.14 mg/mL final concentration) were analyzed using SEC coupled directly to mass spectrometry. Sample injection (1 μL) and on-line SEC-desalting was accomplished using an organic/acidic mobile phase (30% acetonitrile, 0.1% formic acid, 0.02% trifluoroacetic acid) pumped with a Vanquish Horizon UHPLC system (Thermo Scientific) through an SEC UHPLC column (BEH200 SEC 4.6×50 mm, Waters), at a flow rate of 20 μL/min. The LC flow path was connected inline with an Orbitrap Eclipse Tribrid mass spectrometer (Thermo Scientific) via a heated electrospray ionization (HESI-II) ion source (Thermo Scientific). The ion source was operated with 3500 V spray voltage, sheath gas setting of 40 units, auxiliary gas setting of 10 units, and vaporizer temperature of 75° C. The MS capillary temperature was set to 325° C. Intact mass spectra were generated by using an RF Lens setting of 50 V to desolvate protein ions, isolating a mass range of m/z 500-450, and reacting isolated ions with proton transfer charge reduction for 6 ms, followed by scanning for charge reduced intact protein product ions using a mass range of m/z 500-8000. Intact protein ions were detected in the Orbitrap, scanning at a resolution setting of 50,000 (at m/z 200). Raw LC-MS data were analyzed using BioPharma Finder (Thermo Scientific) software. LC-MS spectra representing intact ANXA5-DM1 profiles were deconvoluted using the Respect and Sliding Window algorithms with a ReSpect mass tolerance of 20 ppm, a Sliding Window mass tolerance of 20 ppm, and a deconvolution mass range of 35-45 kDa. Intact mass assignments of individual ANXA5-DM1(n) conjugate isoforms were based on tolerance of 100 ppm. The drug-to-protein ratio was calculated as a weighted average of the ReSpect-Sliding Window abundances for each of the ANXA5-DM1(n) isoforms detected.

### Cell Lines and Culture Conditions

All cell lines and cell media were purchased from American Type Culture Collection (ATCC, Manassas, VA, USA). EMT6 murine breast carcinoma cells were cultivated with Waymouth's MB 752/1 medium (Sigma-Aldrich, St. Louis, MO, USA) supplemented with 2 mM glutamine, 15% FBS, and 1% penicillin/streptomycin antibiotics. 4T1 murine breast cancer cells were cultivated with Roswell Park Memorial Institute 1640 medium (RPMI-1640) supplemented with 10% FBS and 1% penicillin/streptomycin. MCF10A nontumorigenic human breast epithelial cells were cultured in MEGM with MEGM bullet kit (Lonza, Greenwood, SC, USA) (2 mL BPE, 0.5 ml hEGF, 0.5 ml insulin, 0.5 ml hydrocortisone, 0.5 ml GA); GA-1000 was omitted due to ATCC recommendations. Additionally, ATCC recommends 100 ng/ml cholera toxin (Sigma-Aldrich, St. Louis, MO, USA), which was added to the MCF10A cell culture medium as well as 1% antibiotic-antimycotic. All TNBC were passaged using 0.25% (w/v) trypsin in 0.53 mM EDTA (Thermo Fisher Scientific, Waltham, MA, USA) and neutralizing trypsin with fully supplemented medium. MCF10A utilized the same trypsinization procedure but utilized soybean trypsin inhibitor for trypsin neutralization. All cell lines were cultured under a 5% CO₂-supplemented atmosphere at a temperature of 37° C. and 100% relative humidity. The medium was refreshed every 48 h. When plated for studies, adherent cancer cell cultures were grown to less than 75% confluence, and healthy cell lines cells were grown to 100% confluency to mimic the conditions in healthy tissue.

### ANXA5 Colorimetric Binding

Binding strength was analyzed via a modified indirect ELISA. Cells (50,000 cells/well) were grown onto 24 well plates and fixed with 0.025% glutaraldehyde once cancer cells were 70-80% confluent, or MCF10A noncancerous cells were 100% confluent. Cancer cells were blocked with 0.5% BSA for 1 h at 37° C. Cells were incubated with 0-20 nM of biotinylated ANXA5 for 2 h at 37° C. and 5% CO₂. ANXA5 was biotinylated with a Roche biotin protein labeling kit (Roche, Basel, Switzerland), and the level of biotinylation was quantified with a Pierce Biotin Quantification kit (Pierce Biotechnology, Waltham, MA, USA) per manufacturer's instructions. Cells were washed four times with 0.5% BSA to remove unbound ANXA5. Cells were then seeded with 2 μg/ml Strep-HRP for 1 h at room temperature. Cells were washed four times with 0.5% BSA to remove unbound Strep-HRP, and the chromogenic substrate OPD and 30% hydrogen peroxide were added to each well to induce a yellow color change. Cells were incubated at room temperature in the dark. The supernatant was collected, and the absorbance was read at 450 nm on a BioTek Synergy HT microtiter plate reader (Winooski, VT). For each concentration of biotinylated ANXA5, the specific binding was obtained by subtracting non-specific binding, when no calcium was present (biotinylated ANXA5 with 5 mM of EDTA), from the total binding (biotinylated ANXA5 with 2 mM CaCl₂). The dissociation constant was determined using the nonlinear regression one-site total and nonspecific binding model in GraphPad Prism version 9 software (Graph Pad, San Diego California, USA).

### ANXA5-DM1 and ANX5 Cell Viability Assay

Cell viability was assayed by a resazurin assay (AlamarBlue assay, n=3-5). Cancer cells were seeded at 1,000 cells/well in 96 well plates and treated 24-48 h after seeding. Healthy cells were seeded at 10,000 cells per well in 96 well plates and treated once cells reached 90-100% confluency. For ANXA5-DM1 cytotoxicity studies, cells were then treated with 0-100 μM of DM1 in the ANXA5-DM1 conjugate or free DM1 for 72 h in fully supplemented growth medium supplemented with 2 mM calcium to promote ANXA5 binding. For ANXA5 cytotoxicity studies, cells were treated with 0-0.7 μM of ANXA5 for 72 h in fully supplemented growth medium supplemented with 2 mM calcium to promote ANXA5 binding. Following incubation with the drug, cell viability was assayed by resazurin dye reduction assay using AlamarBlue (Thermo Fisher Scientific, Waltham, MA, USA) as per manufacturer instructions and recorded using a Synergy HTX multi-mode microtiter plate reader (BioTek, Winooski, VT, USA). The concentration to inhibit cell growth by 50% (IC50) was derived from the dose-response curves by using the sum of squared differences to fit a sigmoidal regression of the form:

$$V = \frac{Max}{( 1 + \frac{C}{IC 50} ) H}$$

where V is the response (viability), Max is the theoretical maximum response (100% viability), C is the concentration of the drug, and H is the Hill coefficient that describes the “steepness” of the curve.

### Immunogenic Cell Death—ATP Release and Calreticulin Externalization

To determine if ANXA5-DM1 induces ATP release from cells after treatment, an ATP luminescence kit (Molecular Probes, Eugene, OR, USA) was utilized according to the manufacturer's instructions. Briefly, EMT6 and 4T1 cells were seeded at 5,000 cells/well in 96 well plates and treated 24 h after seeding. Cells were treated with 0 or 10 nM of DM1 in the ANXA5-DM1 conjugate or free DM1 for 24 h. After 24 h, 10 μL of cell media was added to 90 μL of standard reaction solution in a white plate. Luminescence reading was obtained from the plate reader, and ATP concentration was calculated by comparing the luminescence reading to a standard curve.

To determine if ANXA5-DM1 induces calreticulin surface expression, flow cytometry was utilized. EMT6 and 4T1 cells were seeded at 25,000 cells/well in 24 well plates and treated 24 h after seeding. Cells were treated with 0 or 10 nM of DM1 in the ANXA5-DM1 conjugate or free DM1 for 24 h. After 24 h, media was collected into microcentrifuge tubes to collect dead cells. To ensure there would be enough cells, three wells were combined to be one sample. Cells in the plates were then washed with PBS, and 100 μL of trypsin was added to each well. Once 90% of the cells were removed from the plate (2-5 min), cells were added to the microcentrifuge tubes and centrifuged at 500×g for 5 min. Cells were resuspended in 200 μL of 0.5% BSA-PBS and counted to have at least 100,000 cells per sample. Cells were then fixed with 4% paraformaldehyde (PFA, Thermo Fisher Scientific, Waltham MA, USA) for 20 min at 4° C. Cells were then washed two times with 500 μl of 0.5% BSA-PBS at 500×g for 5 minutes. After the second wash, cells were resuspended in 100 μl of 0.5% BSA-PBS, and 1 μl of CD16/CD32 Fc Block (eBioscience, San Diego, CA, USA) was added. Cells were then incubated for 20 min at 4° C. Finally, 1 μl of FITC-labeled calreticulin (Novus Biologicals, Centennial, CO, USA) was added. Cells were incubated for another 30 minutes at 4° C. in the dark. After incubation, cells were analyzed on a BD Biosciences Accuri C6 flow cytometer (Franklin Lakes, New Jersey, USA) with excitation at 488 nm, and a 533/30 bandpass filter was used to capture 10,000 gated events per sample. Data was collected and analyzed with BD C6 Accuri software. One sample was composed of three wells, and each sample was analyzed three times. All experiments were conducted in triplicate.

### In Vivo Tumor Models-4T1 and EMT6 Tumor Implantation in BALB cJ Mice

All animal studies were performed in accordance with the protocols approved by the Institutional Animal Care and Use committee (IACUC) at the University of Oklahoma. Animals were housed in a pathogen-free facility at the University of Oklahoma and monitored daily. For 4T1 tumor formation, six-week-old BALB/cJ mice (Jackson Laboratory; 000651) were injected with 1×10⁵ 4T1 cells in the fourth mammary fat. Tumor cell implantation was performed with 30-gauge needle with 50% matrigel (Corning, Corning, NY, USA) and 50% PBS in 100 μL. For EMT6 tumor formation, six-week-old BALB/cJ mice (Jackson Laboratory; 000651) were injected with 1×10⁶ EMT6 cells in the fourth mammary fat. Tumor cell implantation was performed with 30-gauge needle in with cells suspended in 100 μL PBS.

### Mice Mass and Tumor Volume

Mice mass and tumor volumes were measured every 3-7 days. Tumor volumes were measured using caliper measurements, and volume (V) was calculated using the formula V=((L)(W²))/2, where L is the longest diameter of the tumor, and W is the perpendicular diameter of the tumor.

### ANXA5-DM1 Preliminary Dosing

Mice were implanted with 4T1 cells on day 0. Once tumors reached 3-5 mm in diameter, treatment began. Mice were treated with 0-0.25 mg/kg of DM1 intraperitonially (IP) in the ANXA5-DM1 conjugate once every 7 days for 21 days. Tumor volumes and survival were monitored.

### ANXA5-DM1 Holistic Treatment

Mice were implanted with EMT6 or 4T1 cells on day 0. Once tumors reached 3-5 mm in diameter, treatment began. Mice were treated IP with 0.025 mg/kg ANXA5-DM1 once every 7 days, 5 mg/kg, rapamycin daily, and/or 5 mg/kg anti-PD1 on days 1, 5, 9 of treatment. Mice were treated for 21 days, and survival was monitored.

### ANXA5-DM1 Intratumoral Treatment

Mice were implanted with EMT6 cells on day 0. Once tumors reached 3-5 mm in diameter, treatment began. Mice were treated with 0-0.25 mg/kg of DM1 intratumorally (IT) in the ANXA5-DM1 conjugate daily for 21 days, and tumor volumes and survival were monitored.

### ANXA5 Immunogenicity

Protein specific antibody titers were analyzed. Briefly, 3 mg/kg of ANXA5 was administered daily for 21 days. One mouse was euthanized at day 0, 10, and 21 of treatment, and blood was collected via a cardiac puncture. Blood was allowed to coagulate at room temperature for 30 minutes, then centrifuged at 3,500×g for 10 minutes, retaining the supernatant and stored at 80° C. A sandwich ELISA assay was performed to determine protein specific antibody titers. A 0.1 M carbonate coating buffer and 20 μg/mL ANXA5 were incubated overnight at 4° C. on high binding capacity ELISA 96 well plates (VWR; Ranor, PA, USA). Plates were then washed and blocked with fetal bovine serum, and plasma dilutions were incubated overnight at 4° C. Following additional washes, goat anti-mouse IgG and IgM conjugated to HRP (Jackson ImmunoResearch Laboratories, Inc., West Grove, PA; 315-035-044) was used to develop OPD as described in the in vitro binding assays. Antibody titers are presented as the greatest dilution that produces a positive result. ELISA absorbance cut off values were determined by the mean of the negative controls+3 SD, so any absorbance value above this value was considered positive.

### ANXA5-CMB Dosing

Mice were implanted with 4T1 cells on day 0. Once tumors reached 3-5 mm in diameter, treatment began. Mice were treated with 0-5 mg/kg of ANXA5-CMB or free CMB IP daily or on a 5 day on 2 days off dosing cycle that continued for 21 days. Tumor volume and survival were monitored.

### Statistics

All statistical analysis was performed with GraphPad Prism 9 version 4.1. A student's T-test was performed for ANXA5-calcium dependence binding, confluency effects for ANXA5binding, and dose specific cytotoxicity studies that utilized two groups. A one-way analysis of variance (ANOVA) was performed for the in-suspension binding, ANXA5 cytotoxicity, ATP release, calreticulin surface expression, mice mass, and tumor volumes studies that utilized 3 or more groups. Tukey's multiple comparison post hoc analysis was utilized to determine statistical significance between the groups. A log-rank Cox-Mantel test was preformed to analyze survival. A cut off value of p<0.05 indicated significance for all studies.

### Characterization of ANX5-DM1 by Absorbance

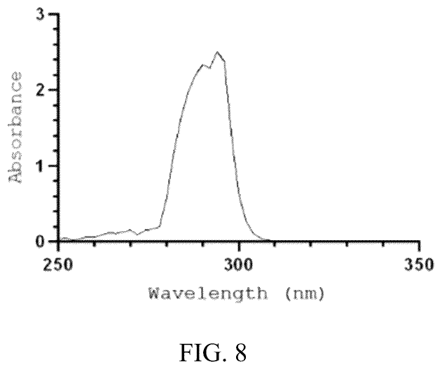

To determine the average number of DM1 molecules per ANXA5 protein, the absorbance of a sample of the conjugate and a sample of the same protein concentration of unconjugated annexin was measured at 288 nm (DM1 peak absorbance). The peaks were subtracted from each other to find the contribution of only DM1 to the absorbance at 288 nm. The resulting absorbance value was compared to a standard curve of DM1 concentrations in solution to determine the concentration of DM1 on the proteins. The molar concentration of DM1 was divided by the molar concentration of the ANXA5 protein to arrive at the average DM1 per ANXA5 loading. A loading of 6:1 DM1 molecules to one ANXA5 molecule was determined. The result of the absorbance versus wavelength after subtraction of the free ANXA5 absorbance is shown in FIG. 8.

### Binding of ANXA5 to Breast Cancer Cells and Healthy Breast Cells

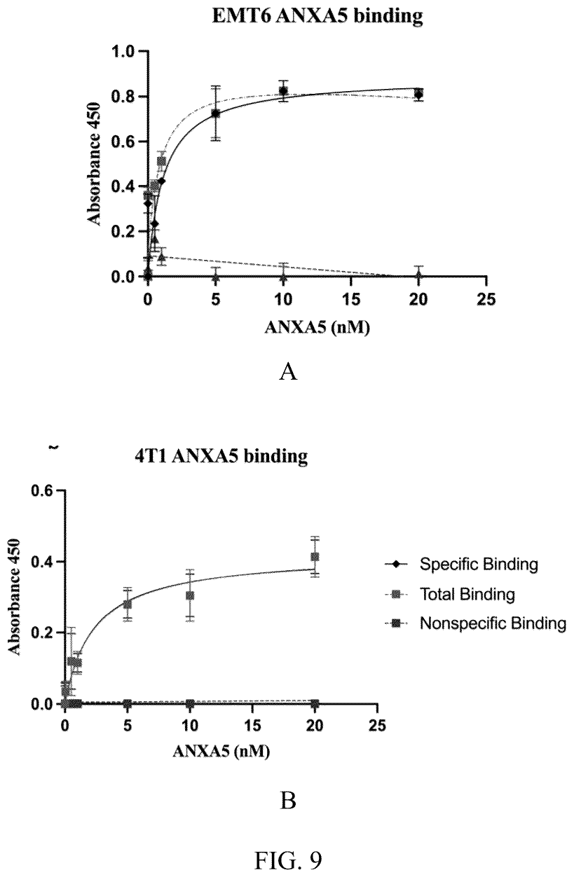

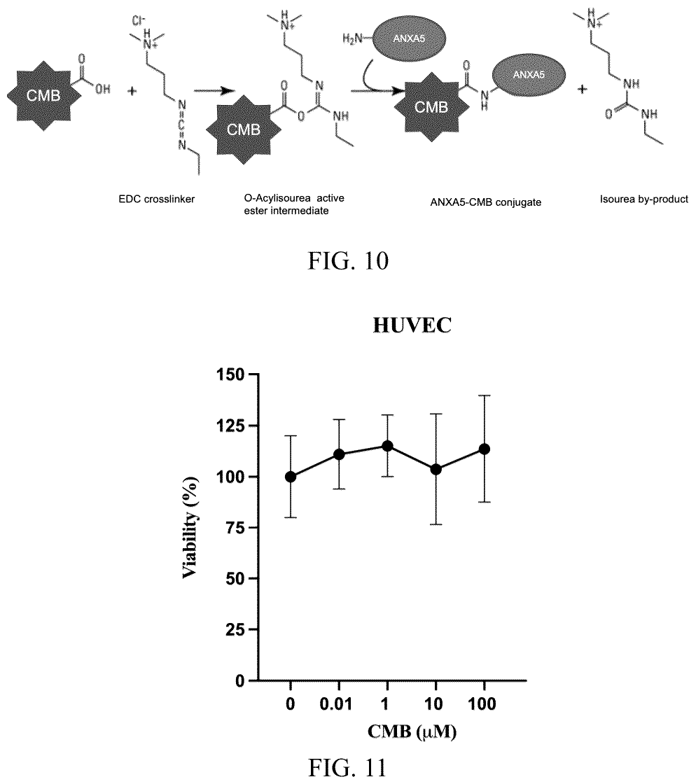

To confirm the ability of the ANXA5 to bind to PS on the surface of TNBC cells, equilibrium binding experiments with increasing concentrations of biotinylated ANXA5 were used. Cells were treated with 0-20 nM of ANXA5, and total, non-specific, and specific binding were obtained. Total binding was obtained by supplementing 0.5% BSA with 2 mM Ca²⁺ to promote ANXA5 binding. Non-specific binding was obtained by supplementing 0.5% BSA with 5 mM EDTA, a calcium-chelating agent. The EDTA removes excess calcium and inhibits ANXA5binding. Specific binding was obtained by subtracting non-specific binding from total binding. After subtracting out 0 nM ANXA5 background absorbance, the non-specific binding was essentially 0, meaning ANXA5 was not binding to the EMT6 and 4T1 cells when Ca²⁺ was absent. The total and specific binding were nearly identical indicating the binding of ANXA5 to PS is calcium-dependent (FIG. 9).

The dissociation constants (K_{d}) for ANXA5 binding to 4T1 cells was 2.31 nM and to EMT6 cells was 1.14 nM was found to be in the low nanomolar range, indicating strong binding. A K_{d} for healthy MCF10A human breast cells grown to confluence was not detectable. For MCF10A cells the total binding and the nonspecific binding overlapped, indicating no specific binding to the cells.

As noted above, ANXA5-DM1 was produced by conjugation using sulfo-SMCC. The ANXA5-DM1 was characterized through absorbance at 288 nm (FIG. 8), SDS-PAGE, and denaturing intact mass analysis. SDS-PAGE showed a molecular weight increase of 6±3 molecules following DM1 (MW 740 Da; MW 960 Da with crosslinker) addition. By deconvoluting the ANXA5-DM1 conjugate in the mass range of 35-45 kDa of denaturing intact mass analysis, a left-shifted bell curve distribution of ANXA5-DM1 is observed. The weighted average of the drug-to-protein ratio obtained was 3.9 molecules of DM1 to 1 molecule of ANXA5, which is within the range previously found with SDS-PAGE analysis.

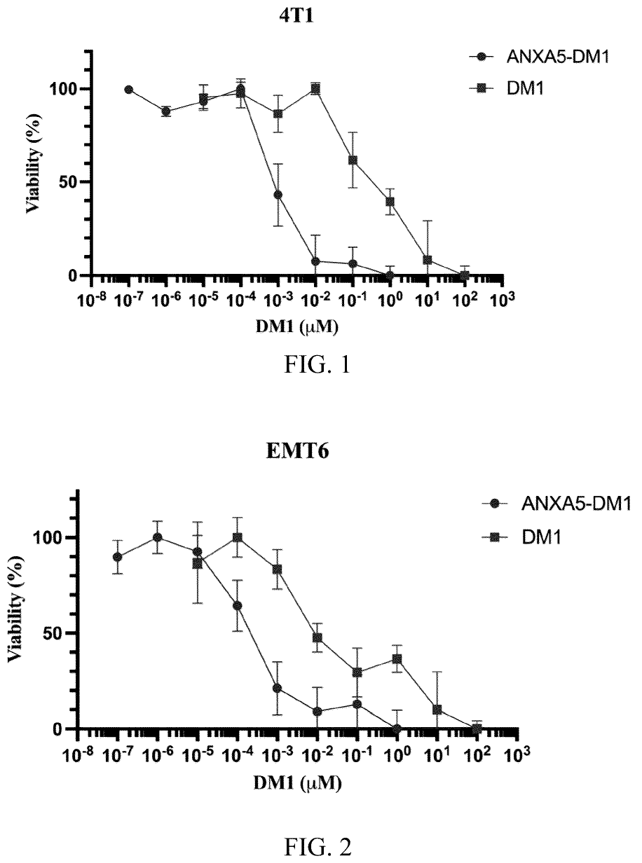

Next, we examined the cytotoxicity against TNBC cells using a 72 h in vitro assay of the ANXA5-DM1 conjugate and free DM1 in mouse 4T1 and EMT6 TNBC cells and in MCF10A healthy mammary cells FIGS. 1-3). For TNBC cells, the antineoplastic activity of DM1 was significantly enhanced as part of an ANXA5 bioconjugate. Measuring the inhibitory concentration of 50 percent (IC50) in a 72 h in vitro viability assay, the ANXA5-DM1 bioconjugate was more than 2 orders of magnitude more potent than free DM1 in the 4T1 and EMT6 cell lines. (FIGS. 1-2, respectively). The ANXA5-DM1 conjugate was significantly less cytotoxic to healthy MCF10A cells grown to 100% confluence than the free DM1 at doses up to 48 μM (FIG. 3). The IC50 of the ANXA5-DM1 could not be determined and exceeded 48 μM; however, the IC50 for free DM1 was 160 nM. Compared to the two TNBC cell lines studied, the 48 μM dose of DM1 in the ANXA5-DM1 conjugate is 56,000 to 229,000 times larger than the IC50 for the noncancerous mammary cells. This indicates the ANXA5-DM1 conjugate is considerably less toxic to healthy breast cells than it is to TNBC cells.

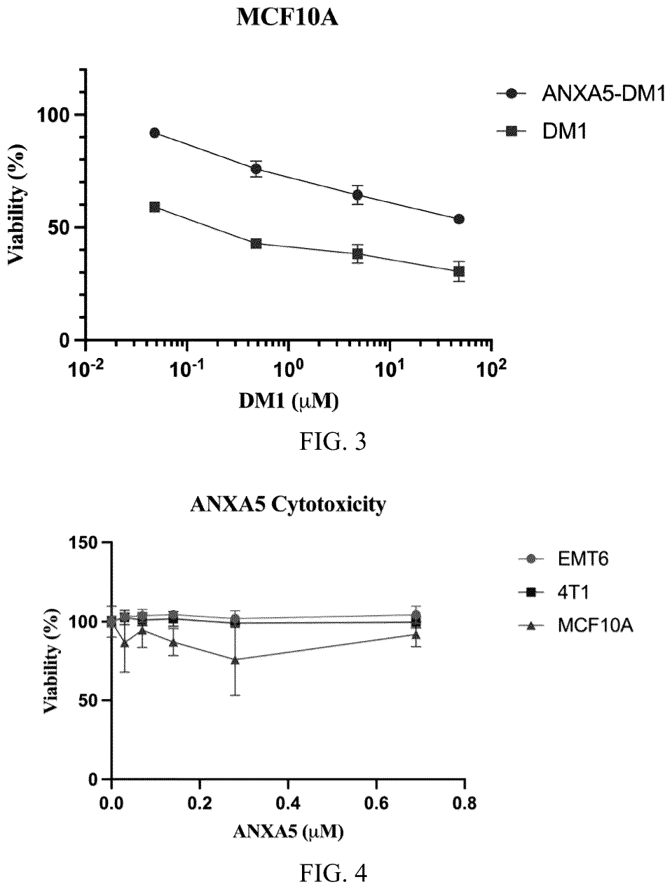

To ensure that DM1, and not ANXA5, is causing the cytotoxic action of the conjugate, cytotoxicity studies of free ANXA5 were carried out on the two TNBC cell line as well as the healthy mammary cell line. The presence of ANXA5 on the two TNBC cell lines and one healthy mammary cell line did not significantly impact viability with doses up to 0.7 μM (FIG. 4). At the higher IC50 of ANXA5-DM1 for the TNBC cells (0.85 nM DM1), the equivalent IC50 based on the ANXA5 concentration is 0.21 nM, assuming 4 moles DM1 per mole ANXA5; this concentration is 3290 times less than the highest ANXA5 concentration tested for free ANXA5. This indicates that the cytotoxic action of the conjugate is a result of the addition of the drug and not the protein alone.

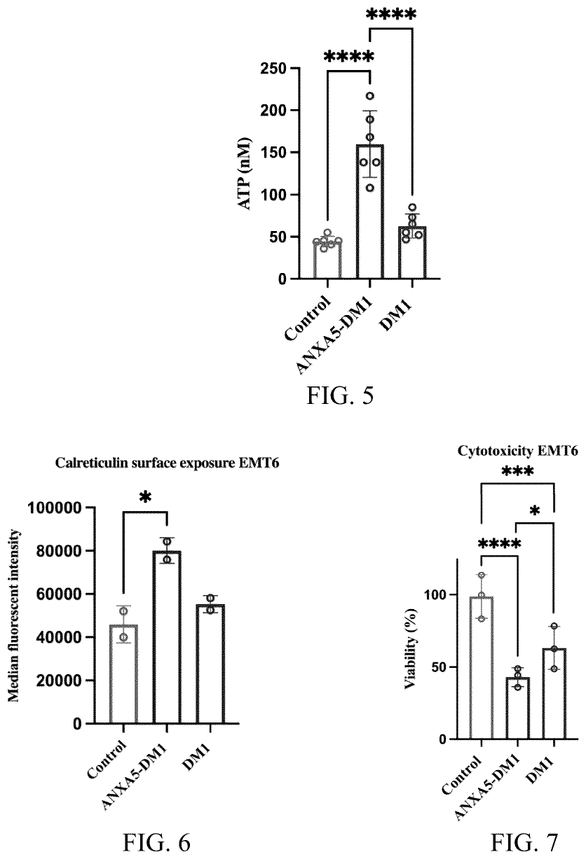

Next, we examined the ability of the ANXA5-DM1 conjugate to induce ICD by evaluating the release of ATP into the extracellular space and calreticulin surface expression. At 10 nM, ANXA5-DM1 significantly increased ATP release (FIG. 5). ANXA5-DM1 caused nearly 10 times more ATP released than free DM1. Cancer cells require large stores of ATP to undergo constant division, protein production, and cell signaling, so, unsurprisingly, the untreated control had essentially 0 ATP in the extracellular space. Incubation with ANXA5-DM1 significantly increased calreticulin surface expression in comparison to the untreated control as determined by median fluorescent intensity (FIG. 6). Although both ANXA5-DM1 and free DM1 significantly increased cell death (FIG. 7), only ANXA5-DM1 significantly increased ICD DAMPs. Additionally, ANXA5-DM1 significantly increased cell death in comparison to free DM1.

FIG. 9 shows binding strength of ANXA5 on EMT6-strain TNBC cells (A) and 4T1-strain TNBC cells (B) with PS externalization. Cells were incubated with 0-20 nM of ANXA5. Total binding was measured with the addition of 2 mM Ca²⁺ to promote ANXA5 binding. Nonspecific binding was measured with the addition of EDTA to chelate residual Ca²⁺, inhibiting ANXA5 binding. Specific binding was obtained by subtracting nonspecific binding from total binding. The dissociation constant of the specific binding was 1.14 nM for EMT6 cells. The dissociation constant of the specific binding was 2.31 nM for 4T1 cells.

In summary, DM1 was conjugated to ANXA5 for the targeted treatment of TNBC. The ANXA5-DM1 conjugate has been shown to quickly initiate death in TNBC cells and to induce two hallmarks of ICD.

### ANXA5-CMB Conjugate

### Chlorambucil (CMB) Mechanism of Action

CMB is an aromatic mustard gas derivative and induces cell death through DNA alkylation. The two electrophilic chlorine atoms typically react with the N7 on guanine in DNA and RNA generating covalent adducts. The adduct then reacts with a neighboring base generating an intrastrand and/or opposite strand crosslink. Once cross-linked the DNA and RNA cannot be opened for translation/transcription, and the cell undergoes apoptosis. While CMB has proven to have activity as an anti-cancer agent, it is untargeted and common side effects are nausea, vomiting, sores/ulcers in the mouth, swollen glands, low white cell count, unusual bleeding/bruising, overall tired/weakness, and changes in menstruation. However, as demonstrated herein, by utilizing the terminal carboxylic acid moiety on CMB, it can be linked to ANXA5, and a targeted treatment of CMB is created.

### Synthesis of ANXA5-CMB Conjugate

The ANAX5-CMB bioconjugate was synthesized by linking CMB (TCI America, Portland, OR, USA) to ANXA5 via 1-ethyl-3(3-dimethylaminopropyl) carbodiimide (EDC) crosslinking chemistry. CMB (6.5 mM) is dissolved in 50 μL of an acid alcohol solution of 3% HCl and 95% EtOH v/v (VWR Inc., Radnor, PA, USA). A working solution is prepared by diluting this acid alcohol solution in 1 mL of 30 mM PBS (Mallinckrodt Chemicals, Phillipsburg, NJ, USA) at pH 5. The carboxylic acid moiety of CMB is then activated with 65 mM EDC, forming an unstable O-acylisourea intermediate for 30 minutes. Excess reactive EDC is quenched with 2 mM B-mercaptoethanol (Sigma-Aldrich, St. Louis, MO, USA), preventing the formation of crosslinked byproducts downstream. The resulting solution of CMB-EDC is added to 2 mL of 27.77 μM (1 mg/ml) ANXA5 in 30 mM PBS, where the activated CMB reacts readily with the

primary amines of ANXA5 lysine residues for 12 h at 4° C. [194]. At this point, excess CMB forms a visible precipitate in neutral pH solution and is removed by centrifuging at 10,000 rcf for 1 h at 4° C. The supernatant is collected, and the resulting ANXA5-CMB bioconjugate is further purified from excess free CMB with an 8-hour dialysis in 2 L of 30 mM sodium phosphate buffer at pH 7.4 with a regenerated cellulose dialysis membrane of MWCO 12-14 kDa (Fisher Scientific, Pittsburgh, PA, USA) at 4° C. Dialysate is changed at least once to ensure removal of unreacted molecules.

### ANXA5-CMB TNBC Cell Cytotoxicity

The ANXA5-CMB conjugate was demonstrated to have enhanced cytotoxicity against murine TNBC cells when compared to free CMB. The IC50 values of the TNBC cells are summarized in Table 1 below. By conjugating CMB to ANXA5, the conjugate was 57 to 241 times more effective at inducing cell death than the free drug after 24 hours. While the cytotoxic activity of the ANXA5-CMB conjugate is excellent against TNBC cells, the cytotoxicity of the conjugate must be tested against healthy vascular and mammary cells.

TABLE 1
Toxicity effects of ANXA5-CMB vs free
CMB IC50 on two murine TNBC cell lines

| Cell Line | ANXA5-CMB IC50 | | CMB IC50 | IC50 ratio |
|---|---|---|---|---|
| 4T1 | 2.7 | μM | 156 μM | 57 |
| EMT6 | 0.42 | μM | 102 μM | 241 |

### ANXA5-CMB Healthy Cell Cytotoxicity

Cell viability was assayed by a resazurin assay (n=3-6). HUVEC and MCF10A cells were seeded at 10,000 cells per well in 96 well plates and treated once cells reached 90-100% confluency. Cells were treated with 0-100 μM of CMB in the ANXA5-CMB conjugate in growth media fully supplemented with 2 mM calcium to promote ANXA5 binding. After 72 hours, an Alamar Blue assay was utilized to determine viability. Percent viability was determined by comparing treatment groups to the untreated control (0 μM).

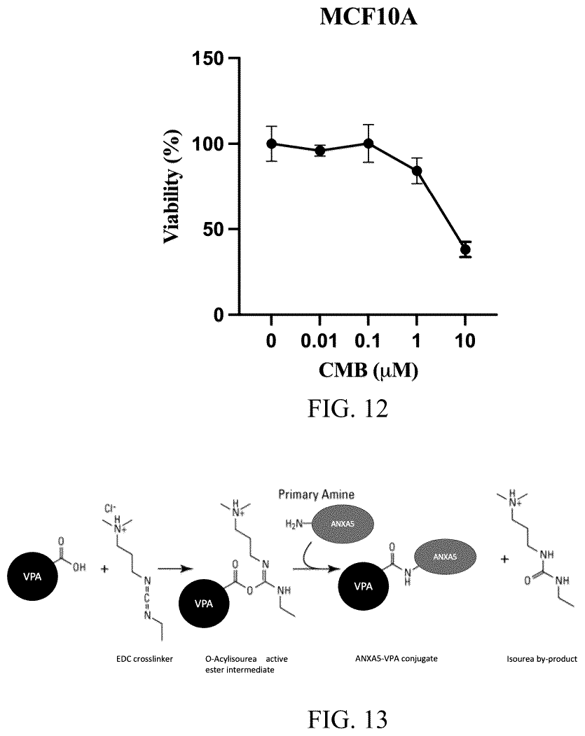

Cytotoxicity of ANXA5-CMB on HUVEC and MCF10A cells is shown in FIGS. 11-12, respectively. The ANXA5-CMB conjugate exhibited 100% cytotoxicity at 1 μM for TNBC cells. The presence of ANXA5-CMB for HUVEC cells did not impact viability with a dose of up to 100 μM (FIG. 11). While the presence of ANXA5-CMB did induce cell death for the MCF10A cells (FIG. 12). The IC50 of ANXA5-CMB for the MCF10A cells was 6.0 μM. When comparing the IC50 for the TNBC cells and that of MCF10A, it takes 2.2 to 14.2 more ANXA5-CMB to induce the same amount of cell death for the healthy MCF10A cells. This indicates ANXA5-CMB uptake is limited in the healthy cell lines, and less cytotoxicity is observed. Ideally, ANXA5-CMB would not have impacted MCF10A viability, but cell death is most likely due to a positive feedback loop as discussed in the ANXA5-DM1 healthy cell cytotoxicity section. Cells were treated with 0-100 μM CMB in the ANXA5-CMB conjugate in fully supplemented growth medium with 2 mM calcium. Cells were grown to 90-100% confluency. ANXA5-CMB did not impact cell viability of HUVEC cells. Cell viability was measured via an Alamar Blue assay and viability was compared to the control (0 μM) after 24 hours. Data is presented as mean±SD (n=4-8). Cells were treated with 0-100 μM CMB in the ANXA5-CMB conjugate in fully supplemented growth medium with 2 mM calcium. Cells were grown to 90-100% confluency. The IC50 of ANXA5-CMB on MCF10A cells was 6.0 μM. Cell viability was measured via an Alamar Blue assay and viability was compared to the control (μM) after 24 hours. Data is presented as mean±SD (n=4-8).

### ANXA5-Valproic Acid Conjugate

### Valproic Acid (VPA) Mechanism of Action

VPA is a class I and class IIa HDACi that leads to hyperacetylation of histones H3 and H4 with favorability at the lysine 9 residue of histone H3 and lysine 8 residue of histone H4. VPA blocks HDAC activity by binding to the catalytic site. More specifically, the carboxylic acid on VPA complexes with the zinc ion in the active site of the HDAC preventing substrate access into the catalytic site. As a free drug, VPA is a potent regulator of gene expression. In cervical cancer, VPA induced a two-fold upregulation of over 1,000 genes, which include genes that regulate cell cycle control, apoptosis, and tumor suppressors. Additionally, a two-fold downregulation of over 500 genes was also observed, which include genes that regulate tumor survival.

In breast cancer, VPA has a wide range of anticancer effects. VPA has been shown to regulate apoptosis by upregulation of P21, Bak, and M30 protein expression, caspase 3 and 7 activations, downregulation of Bcl-2 protein, and to halt cycle progression at the G0/G1 phase. Additionally, VPA has been shown to turn TNBC cells ER-α positive and re-sensitize drug-resistant tumors. While VPA is an excellent candidate as an anticancer drug, when delivered alone, it requires high doses (e.g., 1-10 mM after 72 hours) to be effective in vitro. Additionally, VPA is associated with high liver, heart, and brain metabolism. Additionally, when VPA is in blood, it is bound to albumin, limiting clinical translation for cancer therapy. Because HDAC are overexpressed enzymes that contribute to almost every aspect of cancer growth and progression, creating a targeted treatment of ANXA5-VPA to reset the acetylation homeostasis in TNBC will create the first HDAC-targeted treatment for this orphan disease.

Many chemotherapeutic agents have low aqueous solubility and complex structures that involve multiple carboxylic acids and/or primary amines, making them incompatible with EDC crosslinking. The complex chemistries needed to create unwanted byproducts that decrease the final yield and ultimately lead to an increase in cost. However, the simple structure of VPA enables it to be easily linked to ANXA5. VPA is a short-chain fatty acid with only one carboxylic acid and does not contain any primary amines, meaning unwanted VPA-VPA bioproducts will not be formed. Another advantage of VPA is that it is water soluble, unlike CMB. The ANXA5-VPA conjugation protocol utilizes over 10 times more EDC than the ANXA5-CMB conjugation protocol, and the presence of ANXA5-ANXA5 dimers was not observed with the VPA conjugation. This further validating solubility of activated drug limits drug loading.

### Synthesis of ANXA5-VPA Conjugate

The ANAX5-VPA bioconjugate was synthesized by linking valproic acid, sodium salt (Cayman Chemicals, Ann Arbor, MI, USA) to ANXA5 via EDC crosslinking chemistry. VPA at a concentration of 69.5 mM (10 mg/ml) was dissolved in PBS (pH 7.4). The carboxylic acid moiety of VPA is then activated with 851.6 mM (132 mg/ml) EDC forming an unstable O-acylisourea intermediate for 30 minutes. Excess reactive EDC is quenched with 2 mM β-mercaptoethanol (Sigma-Aldrich, St. Louis, MO, USA), preventing the formation of crosslinked byproducts downstream. The resulting solution of VPA-EDC is added to 1 mL of 27.77 μM (1 mg/ml) ANXA5 in 30 mM PBS, where the activated VPA reacts readily with the primary amines of ANXA5 lysine residues for 2 hours at 25° C. The resulting ANXA5-VPA biconjugate is then purified from excess free VPA with an 8-hour dialysis in 2 L of 30 mM sodium phosphate buffer at pH 7.4 with a regenerated cellulose dialysis membrane of MWCO 12-14 kDa (Fisher Scientific, Pittsburgh, PA, USA) at 4° C. Dialysate is changed at least once to ensure removal of unreacted molecules.

As noted above, the chemical makeup of ANXA5 and VPA make them excellent candidates to be linked together through via EDC crosslinking chemistry (FIG. 13). EDC provides a zero-length linker that reacts carboxylic acids to amines through an addition-elimination reaction. First EDC activates the lone carboxylic acid on VPA creating an unstable o-acylisourea active ester. Next, ANXA5 is introduced, and 1 of the 22 primary amines (lysine residues) displaces the EDC crosslinker, generating a stable amide bond between ANXA5 and VPA. Synthesis of ANXA5-VPA was confirmed with SDS-PAGE analysis. EDC crosslinking does not contribute to the molecular weight of the ANXA5-VPA conjugate, and any increase in weight is a result of the addition of a VPA drug molecule. For every VPA molecule added, a shift upward of 144 Da will be observed. Using gel electrophoresis, we observe a mass increase from 36 kDa of ANXA5 to about 38 kDa which indicates the addition of 14 molecules of VPA to one ANXA5 for two separate conjugations. After quantifying four separate conjugations, the average drug-to-protein ratio of VPA to ANXA5 was about 10:1 (9.7±5.4).

In general, in non-limiting embodiments, the protein-drug conjugates of the present disclosure comprise a plurality of VPA molecules in a range of 5 to 21 VPA molecules per single molecule of annexin protein, or a range of 5 to 20 VPA molecules per single molecule of annexin protein, or a range of 6 to 20 VPA molecules per single molecule of annexin protein, or a range of 6 to 18 VPA molecules per single molecule of annexin protein, or a range of 6 to 16 VPA molecules per single molecule of annexin protein, or a range of 6 to 14 VPA molecules per single molecule of annexin protein, or a range of 7 to 20 VPA molecules per single molecule of annexin protein, or a range of 7 to 18 VPA molecules per single molecule of annexin protein, or a range of 7 to 16 VPA molecules per single molecule of annexin protein, or a range of 7 to 14 VPA molecules per single molecule of annexin protein, or a range of 8 to 20 VPA molecules per single molecule of annexin protein, or a range of 8 to 18 VPA molecules per single molecule of annexin protein, or a range of 8 to 16 VPA molecules per single molecule of annexin protein, or a range of 8 to 14 VPA molecules per single molecule of annexin protein, or a range of 8 to 12 VPA molecules per single molecule of annexin protein, or a range of 9 to 11 VPA molecules per single molecule of annexin protein, or about 10 VPA molecules per single molecule of annexin protein.

In certain embodiments, the present disclosure is directed to a therapeutic composition, comprising (1) a protein-drug conjugate component comprising annexin proteins to which valproic acid molecules are covalently linked, and (2) at least one of an immunostimulant component and an mTOR inhibitor component. The protein-drug conjugate component of the therapeutic composition may comprise an average number in a range of 5 to 21 VPA molecules per molecule of annexin protein, or an average number in a range of 5 to 20 VPA molecules per molecule of annexin protein, or an average number in a range of 5 to 18 VPA molecules per molecule of annexin protein, or an average number in a range of 6 to 20 VPA molecules per molecule of annexin protein, or an average number in a range of 6 to 18 VPA molecules per molecule of annexin protein, or an average number in a range of 6 to 16 VPA molecules per molecule of annexin protein, or an average number in a range of 6 to 14 VPA molecules per molecule of annexin protein, or an average number in a range of 7 to 20 VPA molecules per molecule of annexin protein, or an average number in a range of 7 to 18 VPA molecules per molecule of annexin protein, or an average number in a range of 7 to 16 VPA molecules per molecule of annexin protein, or an average number in a range of 7 to 14 VPA molecules per molecule of annexin protein, or an average number in a range of 8 to 20 VPA molecules per molecule of annexin protein, or an average number in a range of 8 to 18 VPA molecules per molecule of annexin protein, or an average number in a range of 8 to 16 VPA molecules per molecule of annexin protein, or an average number in a range of 8 to 14 VPA molecules per molecule of annexin protein, or an average number in a range of 8 to 12 VPA molecules per molecule of annexin protein, or an average number in a range of 9 to 11 VPA molecules per molecule of annexin protein, or an average of about 10 VPA molecules per molecule of annexin protein.

VPA is already FDA-approved as an antiepileptic drug and can be administered as a free drug. The ANXA5-VPA conjugate can carry over two times more drug (as compared to currently approved protein-dug conjugates) and would possibly not be limited by severe side effects (hair loss, weight loss, nausea, vomiting, etc.) as seen with current chemotherapies and antibody-drug conjugates. The ANXA5-VPA conjugate was relatively pure, with very little ANXA5-ANXA5 crosslinking as indicated by the absence of band smear at approximately 75 kDa. While quantitation limitations exist, the simple chemistry for the ANXA5-VPA is advantageous for yield and cost.

### ANXA5-VPA TNBC Cytotoxicity

After confirming the conjugation of VPA to ANXA5, the cytotoxicity of the ANXA5-VPA conjugate was tested in two murine TNBC cell lines (EMT6 and 4T1). Cell viability was assayed by a resazurin assay (n=3). EMT6 and 4T1 cells were seeded at 5,000 cells/well in 96 well plates and treated 48 hours after seeding. Cells were treated with 10⁻⁴ to 10 mM of VPA in the ANXA5-VPA conjugate or free VPA growth medium fully supplemented with 2 mM of calcium to promote ANXA5 binding. After 48 hours, cell viability was analyzed via a fluorescent Alamar Blue assay. Percent viability was determined by normalizing the control (0 mM VPA) to 100% and comparing each treatment group to the control (n=3).

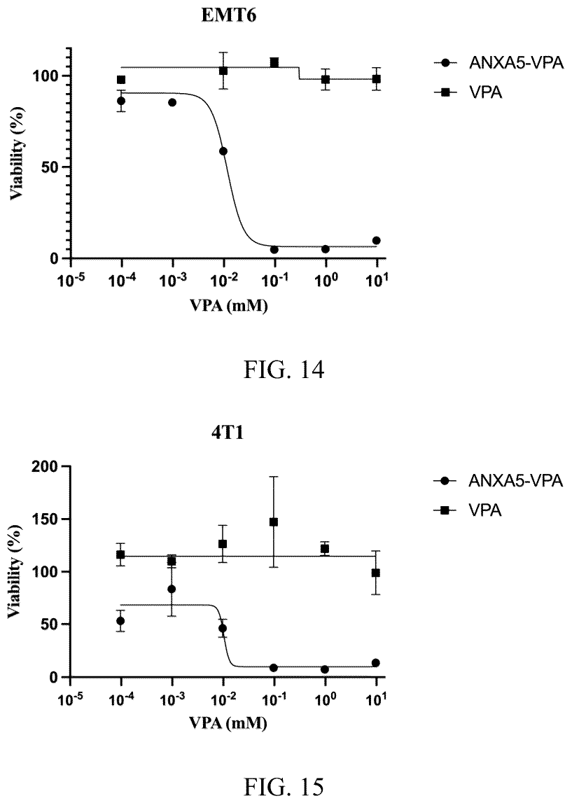

For both murine TNBC cell lines, the ANXA5-VPA conjugate showed superior cytotoxic activity when compared to free VPA after 48 hours (FIGS. 14-15). The ANXA5-VPA conjugate significantly increased cell death at doses as low as 97 nM when compared to the free drug control. The IC50 after 48 hours for both cell lines was approximately 10⁻² mM, but the IC50 value for free VPA could not be obtained with a dose of up to 10 mM. This finding for free VPA is unsurprising, given the fact that VPA requires over 1 mM and 72-96 hours to achieve an IC50 value. The ANXA5-VPA conjugate not only decreases the concentration of VPA needed to induce cell death but also decreases time. To our best knowledge, the presently disclosed conjugate is the first protein-VPA conjugate to be developed. A tumor-homing peptide-VPA conjugate was previously developed (Peng et al., Bioorg Med Chem Lett. 2014 Apr. 15; 24 (8): 1928-1933) which comprised three parts, (1) the tumor-homing peptide iRGD, a cyclic cell-penetrating peptide that is localized at integrins expressed on tumor vasculature, (2) a lysosomal degrading tetrapeptide spacer which allows the drug to be released into the cell, and (3) a single molecule of valproic acid. The peptide-VPA conjugate was formed using a six-step reaction involving solid-phase peptide synthesis and purified via semi-preparative HPLC. In comparison, the disclosed ANXA5-VPA conjugate requires only two steps and has a high overall yield. The peptide-VPA conjugate was tested on prostate cancer cells but required a full 72 hours and a VPA dose above 1 mM to be effective. More importantly, the covalent addition of VPA did not affect cytotoxicity differently than a mixture of VPA with the peptide component, whereas the cytotoxic effect of the ANXA5-VPA conjugate is a result of the covalent addition of VPA and not the protein. Additionally, the ANXA5-VPA conjugate delivers approximately 10 times more VPA than the peptide-VPA conjugate, and internalization is nonreceptor mediated, unlike iRGD.

While the present disclosure has been described in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the presently disclosed methods and compositions. Changes may be made in the formulation of the various compositions described herein, the methods described herein or in the steps or the sequence of steps of the methods described herein without departing from the spirit and scope of the present disclosure.

What is claimed is:

1. A protein-drug conjugate, comprising:
an annexin protein to which a plurality of valproic acid (VPA) molecules are covalently linked.

2. The protein-drug conjugate of claim 1, wherein the annexin protein is human annexin A5.

3. The protein-drug conjugate of claim 1, wherein the VPA molecules are covalently linked to the annexin protein by using 1-ethyl-3(3-dimethylaminopropyl) carbodiimide (EDC) crosslinking chemistry.

4. The protein-drug conjugate of claim 1, wherein the plurality of VPA molecules is in a range of 5 to 20 VPA molecules per molecule of annexin protein.

5. A therapeutic composition, comprising:
(1) a protein-drug conjugate component comprising annexin proteins to which valproic acid molecules are covalently linked; and
(2) at least one of an immunostimulant component and an mTOR inhibitor component.

6. The therapeutic composition of claim 5, wherein the immunostimulant component is selected from the group consisting of cyclophosphamide, glycated chitosans, muramyldipeptide derivatives, trehalose-dimycolates, BCG-cell wall skeletons, cytokines, and combinations thereof.

7. The therapeutic composition of claim 5, wherein the mTOR inhibitor component is selected from the group consisting of rapamycin, everolimus, temsirolimus, ridaforolimus, metformin, tacrolimus, ABT-578, AP23675, AP-23841, 7-epi-rapamycin, 7-thiomethyl-rapamycin, 7-epi-tromethoxyphenyyl-rapamycin, 7-epi-thiomethyl-rapamycin, 7-demethoxy-rapamycin, 32-demethoxy-rapamycin, 7-desmethyl-rapamycin, 42-O-(2-hydroxy) ethyl-rapamycin, and other analogs of rapamycin.

8. The therapeutic composition of claim 5, wherein the annexin proteins comprise human annexin A5.

9. The therapeutic composition of claim 5, wherein the protein-drug conjugate component comprises an average number of 5 to 20 VPA molecules per molecule of annexin protein.

10. A method of treating a subject afflicted with breast cancer characterized by a triple-negative phenotype, comprising:
administering to the subject a therapeutic amount of a protein-drug conjugate comprising an annexin protein to which a plurality of valproic acid molecules are covalently linked.

11. The method of claim 10, wherein the annexin protein is human annexin A5.

12. The method of claim 10, wherein the plurality of VPA molecules is in a range of 5 to 20 VPA molecules per molecule of annexin protein.

13. The method of claim 10, further comprising administering a therapeutically-effective amount of at least one of an immunostimulant and an mTOR inhibitor to the subject.

14. The method of claim 13, wherein the immunostimulant is selected from the group consisting of cyclophosphamide, glycated chitosans, muramyldipeptide derivatives, trehalose-dimycolates, BCG-cell wall skeletons, cytokines, and combinations thereof.

15. The method of claim 13, wherein the mTOR inhibitor is selected from the group consisting of rapamycin, everolimus, temsirolimus, ridaforolimus, metformin, tacrolimus, ABT-578, AP23675, AP-23841, 7-epi-rapamycin, 7-thiomethyl-rapamycin, 7-epi-tromethoxyphenyyl-rapamycin, 7-epi-thiomethyl-rapamycin, 7-demethoxy-rapamycin, 32-demethoxy-rapamycin, 7-desmethyl-rapamycin, 42-O-(2-hydroxy) ethyl-rapamycin, and other analogs of rapamycin.

16. A protein-drug conjugate, comprising:
human annexin A5 protein; and
a plurality of valproic acid molecules in a range of 5 to 20 VPA molecules per molecule of annexin protein;
wherein the plurality of valproic acid molecules are covalently linked to the human annexin A5 protein via 1-ethyl-3(3-dimethylaminopropyl)carbodiimide (EDC) crosslinking chemistry.
